(19) United States
(12) Patent Application Publication
(10) Pub. No.: US 2022/0217956 A1
(43) Pub. Date: Jul. 14, 2022

(54) Rodent Model Of Increased Bone Mineral Density

(71) Applicant: Regeneron Pharmaceuticals, Inc., Tarrytown, NY (US)

(72) Inventors: Yajun Tang, Tarrytown, NY (US); Alan Shuldiner, Tarrytown, NY (US); Cristopher Van Hout, Tarrytown, NY (US)

(21) Appl. No.: 17/708,198

(22) Filed: Mar. 30, 2022

Related U.S. Application Data
(63) Continuation of application No. PCT/US20/58665, filed on Nov. 3, 2020.
(60) Provisional application No. 62/930,138, filed on Nov. 4, 2019.

(51) Int. Cl. A01K 67/027; A61P 19/08; C12N 15/85

(52) U.S. Cl. CPC A01K 67/0276; A61P 19/08; C12N 15/8509; A01K 2217/077; A01K 2217/072; A01K 2217/15; A01K 2207/12; A01K 2227/105

(57) ABSTRACT

The present disclosure provides rodent models of increased bone mineral density and/or bone mineral content, genetically modified rodents and isolated rodent cells or tissues having a disruption of one or both alleles of the Zinc and Ring Finger 3 (Znrf3) gene, knockout rodent Znrf3 DNA constructs, methods of producing genetically modified rodents, methods of producing Znrf3 knockout rodents, and methods of determining the effect of an agent for treating low bone mineral density and/or bone mineral content.

### REFERENCE TO SEQUENCE LISTING

This application includes a Sequence Listing filed electronically as a text file named 18923803002SEQ, created on Oct. 30, 2020, with a size of 31 kb. The Sequence Listing is incorporated herein by reference.

### FIELD

The present disclosure is directed to rodent models of increased bone mineral density, genetically modified rodents and isolated rodent cells or tissues having a disruption of one or both alleles of the Zinc and Ring Finger 3 (Znrf3) gene, knockout rodent Znrf3 DNA constructs, methods of producing genetically modified rodents, methods of producing Znrf3 knockout rodents, and methods of determining the effect of an agent for treating high bone mineral density.

### BACKGROUND

Increased bone mineral content can be caused by a wide variety of conditions and may result in significant medical problems.

Zinc And Ring Finger 3 (ZNRF3) is an E3 ubiquitin-protein ligase that acts as a negative regulator of the Wnt signaling pathway by mediating the ubiquitination and subsequent degradation of Wnt receptor complex components Frizzled and LRP6. Homologous proteins are also found in mice and rats. ZNRF3 acts on both canonical and non-canonical Wnt signaling pathway. ZNRF3 also acts as a tumor suppressor in the intestinal stem cell zone by inhibiting the Wnt signaling pathway, thereby restricting the size of the intestinal stem cell zone.

### SUMMARY

The present disclosure provides genetically modified rodents having a genetic modification comprising a disruption of one or both alleles of the Zinc and Ring Finger 3 (Znrf3) gene, wherein when at least one allele is disrupted, the rodent exhibits increased bone mineral content (BMC) and bone volume compared to a wild type rodent in which neither Znrf3 allele is disrupted.

The present disclosure also provides conditional knockout rodents having a genetic modification comprising at least one mutant Znrf3 allele that comprises, from 5′ to 3′, a first loxP site, a first FLP recombinase target (FRT) sequence, a reporter gene coding sequence, a second FRT sequence, a rodent Znrf3 cDNA coding sequence, and a second loxP site, wherein: when an FLP recombinase is provided by a genetic cross with an FLP recombinase-expressing rodent, the ends of the first FRT and the second FRT are exchanged such that the reporter gene coding sequence is deleted, and the rodent Znrf3 cDNA coding sequence is rescued; and when a Cre-recombinase is provided by a genetic cross with a Cre-expressing rodent, the rodent Znrf3 cDNA coding sequence is deleted resulting in the absence of rodent Znrf3 cDNA, and the rodent exhibits increased BMC and bone volume compared to a wild type rodent in which Znrf3 cDNA coding sequence is not deleted.

The present disclosure also provides isolated rodent cells or tissues having a genetic modification comprising a disruption of one or both alleles of the Zinc and Ring Finger 3 (Znrf3) gene.

The present disclosure also provides isolated rodent cells or tissues having a genetic modification comprising at least one mutant Znrf3 allele that comprises, from 5′ to 3′, a first loxP site, a first FLP recombinase target (FRT) sequence, a reporter gene coding sequence, a second FRT sequence, a rodent Znrf3 cDNA coding sequence, and a second loxP site, wherein: when an FLP recombinase is provided by a genetic cross with an FLP recombinase-expressing rodent, the ends of the first FRT and the second FRT are exchanged such that the reporter gene coding sequence is deleted, and the rodent Znrf3 cDNA coding sequence is rescued; and when a Cre-recombinase is provided by a genetic cross with a Cre-expressing rodent, the rodent Znrf3 cDNA coding sequence is deleted resulting in the absence of rodent Znrf3 cDNA, and the rodent exhibits increased BMC and bone volume compared to a wild type rodent in which Znrf3 cDNA coding sequence is not deleted.

The present disclosure also provides knockout rodent Znrf3 DNA constructs comprising a selectable marker sequence or a reporter gene, or both, flanked by DNA sequences homologous to rodent Znrf3 genomic DNA, wherein when the construct is introduced into an embryonic rodent cell, the selectable marker sequence disrupts the rodent Znrf3 gene in the embryonic cell, and the rodent resulting from the embryonic cell exhibits increased BMC and bone volume compared to a wild type rodent in which the Znrf3 gene is not disrupted; and vectors comprising the same.

The present disclosure also provides conditional knockout rodent Znrf3 DNA constructs comprising, in the 5′ to 3′ direction: a first rodent Znrf3 genomic DNA fragment; a first loxP site; a first FRT sequence; a reporter gene coding sequence; a second FRT sequence; a rodent Znrf3 cDNA coding sequence; a second loxP site; and a second rodent Znrf3 genomic DNA fragment; and vectors comprising the same.

The present disclosure also provides methods of producing a genetically modified rodent, comprising: transforming a rodent embryonic stem cell with a knockout construct comprising a selectable marker sequence and/or a reporter gene sequence, flanked by DNA sequences homologous to the endogenous rodent Znrf3 genomic DNA, thereby producing a transformed embryonic stem cell; introducing the transformed embryonic stem cell into a rodent blastocyst; and implanting the blastocyst comprising the transformed embryonic stem cell into a pseudopregnant female rodent, and allowing the blastocyst to undergo fetal development to term, to produce the genetically modified rodent; wherein the genetically modified rodent is a heterozygous knockout rodent and exhibits increased BMC and bone volume compared to a wild type rodent.

The present disclosure also provides methods of producing a Znrf3 knockout rodent having a genome which is homozygous fora disruption of the rodent Znrf3 gene, the method comprising: breeding a first heterozygous knockout rodent produced in accordance with any one of claims 61 to 65 with a second heterozygous knockout rodent to produce a progeny rodent; and selecting a progeny rodent in which the disruption of the Znrf3 gene is homozygous.

The present disclosure also provides methods of determining the effect of an agent for treating high bone mineral density, the method comprising: administering the agent to a rodent that is heterozygous or homozygous for a Znrf3 gene knockout; subjecting the rodent to a test to assess bone mineral density; and determining whether the agent has any effect on the bone mineral density in the rodent.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows that Znrf3 heterozygous null mice have increased bone mineral content and increased bone volume; % Difference ((KO−WT)/WT×100); (body weight=−3.82; bmc=+8.96; bmd=+1.47; bone volume=+7.36; bone volume perc.=+12.73; fat volume=−42.68; fat volume perc.=−36.24; lean volume=+3.83; lean volume perc.=+9.29).

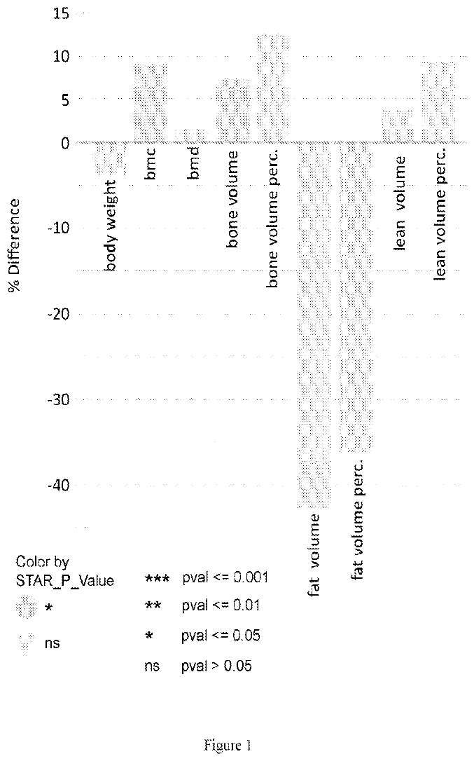

FIG. 2A shows that Znrf3 heterozygous null mice have increased bone mineral content.

FIG. 2B shows that Znrf3 heterozygous null mice have increased bone volume.

### DESCRIPTION OF EMBODIMENTS

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the term “comprising” can be replaced with “consisting” or “consisting essentially of” in any of the embodiments described herein.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “knockout rodent” (or “KO rodent”) is a rodent having a genome in which a particular endogenous gene has been inactivated, such as by the method of gene targeting. A knockout rodent can be a heterozygote (i.e., having a single defective/disrupted allele and one wild type allele) or a homozygote (i.e., having two defective/disrupted alleles and no wild type allele). “Knockout” of an endogenous target gene means an alteration in the sequence of the endogenous gene resulting in a decrease in the function or, more commonly, loss of function of the target gene. In some embodiments, the target gene expression is undetectable or insignificant. A knockout of a Znrf3 gene means that function of the Znrf3 gene has been substantially decreased or lost so that Znrf3 expression is not detectable (or may only be present at insignificant levels). The term “knockout” is intended to include partial or complete reduction of the expression of at least a portion of a polypeptide encoded by the targeted endogenous gene (here Znrf3) of a single cell, a population of selected cells, or all the cells of a rodent.

As used herein, the terms “rodent” and “rodents” mean all members of the phylogenetic order Rodentia including any and all progeny of all future generations derived therefrom and include mice and rats.

As used herein, the term “mouse” means all members of the family Muridae, primarily mice.

As used herein, the term “Znrf3-associated disorder” means any physiological state or pathological condition or disease associated with altered Znrf3 function (e.g., due to aberrant Znrf3 expression, usually under-expression, or a defect in Znrf3 expression or in the ZNRF3 protein). Znrf3-associated disorders include, but are not limited to, disorders associated with increased Znrf3 protein resulting in a phenotype characterized by increased BMC, bone mineral density, and bone volume, and include, but are not limited to, osteoarthritic spondylosis, diffuse idiopathic skeletal hyperostosis (DISH), ankylosing spondylitis, Paget's disease, tuberous sclerosis, osteosclerosis, synovitis/acne/pustulosis/hyperostosis/osteitis (SAPHO) syndrome, renal osteodystrophy, acromegaly, Hepatitis C-associated osteosclerosis, myelofibrosis, and mastocytosis.

The mouse Znrf3 gene (GenBank Accession No. NM_001290501) is located on chromosome 11, and is documented as having 9 exons, with exon 1 being the first coding exon. Exemplary mouse cDNA and protein sequences are set forth in SEQ ID NO:1 (NM_001290501.1) and SEQ ID NO:3 (NP_001277430), respectively.

The rat Znrf3 gene (GenBank Accession No. XM_017599550) is located on chromosome 14, and is documented as having 9 exons, with exon 1 being the first coding exon. Exemplary rat cDNA and protein sequences are set forth in SEQ ID NO:2 (XM_017599550.1) and SEQ ID NO:4 (XP 017455039.1), respectively.

ZNRF3 is highly conserved across species, with the mouse and rat proteins being 85.3% identical, and with the mouse and rat proteins being 85.2% and 78.3% identical to the human protein, respectively.

A rare variant in the human ZNRF3 gene associated with a decreased risk of developing decreased bone mineral density or conditions resulting from decreased bone mineral density in human subjects has been identified. For example, a genetic alteration that results in the deletion of a guanine at position 167,122 in the human ZNRF3 reference gene, or a genetic alteration that results in replacement of the adenine at position 166,500 in the human ZNRF3 reference gene with guanine, has been observed to indicate that the human having such an alteration may have a decreased risk of developing decreased bone mineral density or conditions resulting from decreased bone mineral density. Accordingly, the present disclosure provides knockout rodents, and methods of producing the same and using the same, as models for bone mineral density and/or conditions resulting from increased bone mineral density. Accordingly, genetically modified rodents are described herein which are suitable for use as animal models of increased bone mineral density.

The present disclosure provides genetically modified rodents having a genetic modification comprising a disruption of one or both alleles of the Zinc and Ring Finger 3 (Znrf3) gene. In these rodents, when at least one allele is disrupted, the rodents exhibit increased BMC and bone volume compared to wild type rodents in which neither Znrf3 allele is disrupted. A disruption of one or both alleles of the Znrf3 gene includes the deletion of or replacement by an exogenous nucleotide sequence (i.e., a knockout construct) into a homologous endogenous region of the coding region(s) of the endogenous Znrf3 gene of the rodent and/or the promoter region of this gene resulting in the decrease or prevention of the expression of the full length Znrf3 protein in the rodent cell. For example, when such a knockout construct is inserted into an ES cell, the exogenous nucleotide sequence integrates into the genomic DNA of at least one Znrf3 allele to produce a transformed cell. Progeny cells of the of the transformed cell will no longer express Znrf3 or will express it at a decreased level and/or in a truncated or other mutated form, as the endogenous coding region of Znrf3 is now disrupted by the knockout construct.

In some embodiments, the genetically modified rodent comprises an endogenous rodent Znrf3 gene that lacks a portion of the wild type rodent Znrf3 gene. For example, insertion of a knockout construct into an endogenous Znrf3 gene results in replacement of at least a portion of the endogenous Znrf3 gene sequence. In some embodiments, the genetically modified rodent comprises an endogenous rodent Znrf3 gene that lacks a fragment beginning from the nucleotide after the start codon through a subsequent coding exon from the wild type rodent Znrf3 gene. In some embodiments, the rodent genomic fragment being replaced comprises the ATG start codon (in the first coding exon) through the stop codon (in the last coding exon) of the endogenous rodent Znrf3 gene. In some embodiments, the rodent genomic fragment being replaced further comprises a 5′ non-coding exonic sequence or a 3′ non-coding exonic sequence of a rodent Znrf3 gene, or a combination thereof. In some embodiments, the rodent genomic fragment being replaced also comprises the 5′ non-coding sequence in exon 1, or the 3′ UTR of exon 9 of the endogenous Znrf3 gene, or a combination thereof. In some embodiments, the rodent genomic fragment being replaced comprises a fragment beginning from the nucleotide immediately after the start codon in the first coding exon through a subsequent coding exon (e.g., the second, third, fourth, fifth, sixth, seventh, eighth, or ninth coding exon).

In some embodiments, the genetically modified rodents comprise a genetically modified Znrf3 gene, wherein the genetic modification comprises a replacement of all or part of the endogenous rodent Znrf3 gene with a reporter gene, which forms part of the knockout construct. In some embodiments, the reporter gene is operably linked to the endogenous rodent Znrf3 promoter. In some embodiments, the reporter gene is inserted immediately downstream of the start codon (and is operably linked thereto) of the endogenous rodent Znrf3 gene. In such linkage, expression of the reporter gene is expected to resemble the expression pattern of an unmodified endogenous rodent Znrf3 gene.

Any reporter gene can be used. In some embodiments, the reporter gene is a LacZ gene. In some embodiments, the reporter gene is a gene encoding a protein selected the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), Discosoma sp. red fluorescent protein (DsRed), and Meandrina meandrites green fluorescent protein (MmGFP).

In some embodiments, the genetically modified rodent is homozygous for the genetic modification. In some embodiments, the genetically modified rodent is heterozygous for the genetic modification.

In some embodiments, the rodents disclosed herein are incapable of expressing an endogenous rodent ZNRF3 protein. For example, when a genomic fragment in each of the two endogenous rodent Znrf3 alleles has been partially or fully deleted or replaced with knockout construct sequences, thereby resulting in two disrupted Znrf3 alleles, such a rodent is incapable of expressing an endogenous rodent ZNRF3 protein. In some embodiments, when a genomic fragment of one endogenous rodent Znrf3 allele has been replaced with a reporter gene, and the other endogenous rodent Znrf3 allele has been modified to contain a disruption or deletion, the resulting rodent is incapable of expressing an endogenous rodent ZNRF3 protein.

In any of the embodiments described herein, the rodents can be mice, rats, or hamsters.

In some embodiments, the genetically modified rodent is a mouse. In some embodiments, the rodent is a mouse of a CS7BL strain, for example, a CS7BL strain selected from C57BL/A, C57BL/An, C57BL/GrFa, C57BL/KaLwN, C57BL/6, C57BL/6J, C57BL/6ByJ, CS7BL/6NJ, C578L/10, C57BL/10ScSn, C57BL/10Cr, and C57BL/Ola. In some embodiments, the rodent is a mouse of a 129 strain, for example, a 129 strain selected from the group consisting of 129P1, 129P2, 129P3, 129X1, 129S1 (e.g., 129S1/SV, 129S1/Svlm), 129S2, 129S4, 129S5, 129S9/SvEvH, 129/SvJae, 129S6 (129/SvEvTac), 129S7, 129S8, 129T1, 129T2. In some embodiments, the rodent is a mouse that is a mix of an aforementioned 129 strain and an aforementioned C57BL/6 strain. In some embodiments, the mouse is a mix (i.e., hybrid) of a 129 strain mouse, or a mix of a C57BL strain mouse, or a mix of a C57BL strain and a 129 strain. In some embodiments, the mouse is a mix of a C57BL/6 strain with a 129 strain. In some embodiments, the mouse is a VGF1 strain, also known as F1H4, which is a hybrid of C57BL/6 and 129. In some embodiments, the mouse is a BALB strain, e.g., BALB/c strain. In some embodiments, the mouse is a mix of a BALB strain and any other mouse strain.

In some embodiments, the genetically modified rodent is a rat. In some embodiments, the rat is selected from a Wistar rat, an LEA strain, a Sprague Dawley strain, a Fischer strain, F344, F6, and Dark Agouti. In some embodiments, the rat is a mix of two or more strains selected from the group consisting of Wistar, LEA, Sprague Dawley, Fischer, F344, F6, and Dark Agouti.

The present disclosure also provides an approach to conditionally disrupt an endogenous Znrf3 gene in a specific organ or tissue, such as bone, lung, or kidney, by crossing Znrf3 conditional knockout mice with mice that carry a tissue-specific Cre transgene. This approach enables analysis of the role of Znrf3 in those organs over a prolonged period.

In some embodiments, “conditional knockouts”, by inclusion of certain sequences in or surrounding the altered target, make it possible to control whether or not the target gene is rendered nonfunctional. This control can be exerted by exposure of the animal to a substance that promotes target gene alteration, introduction of an enzyme that promotes recombination at the target gene site (e.g., Cre in the Cre-lox system), or any other method that directs or controls the target gene alteration postnatally. Cre recombinase is a 34 kDa protein that catalyzes recombination between two of its recognition sites called loxP. The loxP site is a 34 base pair consensus sequence consisting of a core spacer sequence of 8 base pairs and two flanking 13 base pair palindromic sequences. One of the advantages to this system is that there is no need for additional co-factors or sequence elements for efficient recombination regardless of cellular environment. Recombination occurs within the spacer area of the loxP sites. The post-recombination loxP sites are formed from the two complementary halves of the pre-recombination sites. The result of the Cre recombinase-mediated recombination depends on the location and orientation of the loxP sites. When an intervening sequence is flanked by similarly oriented loxP sites, as in the present invention, Cre recombinase activity results in excision.

Conditional knockouts of the Znrf3 gene function are also included within the present disclosure. Conditional knockouts are transgenic animals that exhibit a defect in Znrf3 gene function. For example, a rodent having a conditional knockout of Znrf3 gene function can be produced using the Cre-loxP recombination system (see, e.g., Kilby et al., Trends Genet., 1993, 9, 413-421). Cre is an enzyme that excises the DNA between two recognition sequences, termed loxP. This system can be used in a variety of ways to create conditional knockouts of rodent Znrf3. For example, in addition to a mouse in which the Znrf3 sequence is flanked by loxP sites, a second mouse transgenic for Cre is produced. The Cre transgene can be under the control of an inducible, or developmentally regulated promoter (Gu et al., Cell, 1993, 73, 1155-1164; Gu et al., Science, 1994, 265, 103-106), or under control of a tissue-specific or cell type-specific promoter (e.g., a pancreas-specific promoter or brain tissue-specific promoter). The Znrf3 transgenic rodent is then crossed with the Cre transgenic rodent to produce progeny rodent deficient for the Znrf3 gene only in those cells that expressed Cre during development. Examples of producing conditional KO mice can be found in, for example, U.S. Patent Application Publications: 2004/0045043, 2004/0241851, and 2006/0064769.

In addition, the FLP-FRT system (see, for example, Dymecki, Proc. Nat'l. Acad. Sci., 1996, 93, 6191-6) has become more commonly used, primarily in work with mice. It is similar to the Cre-Lox system in many ways, involving the use of “flippase” (FLP) recombinase, derived from the yeast Saccharomyces cerevisiae and native to the 2 micron plasmid resident in these yeast cells. In lieu of loxP sites, FLP recognizes a pair of FLP recombinase target (“FRT”) sequences flanking the genomic region of interest. As with loxP sites, orientation of the FRT sequences dictates inversion or deletion events in the presence of FLP recombinase. The FLP recombinase is active at a particular 34 base pair DNA sequence, termed the FRT (FLP recombinase target) sequence. When two FRT sites are present, the FLP enzyme creates double-stranded DNA breaks, exchanges the ends of the first FRT with those of the second target sequence, and then re-attaches the exchanged strands. This process leads to inversion or deletion of the DNA which lies between the two sites. Whether an inversion or deletion occurs depends on the orientation of the FRT sites: if the sites are in the same orientation, the intervening DNA is deleted, but if the sites are opposite in orientation, the DNA is inverted. The FLP recombinase is used as a negative selectable marker for experiments to replace genes by homologous recombination.

The present disclosure also provides conditional knockout rodents having a genetic modification comprising at least one mutant Znrf3 allele that comprises, from 5′ to 3′, a first loxP site, a first FLP recombinase target (FRT) sequence, a reporter gene coding sequence, a second FRT sequence, a rodent Znrf3 cDNA coding sequence, and a second loxP site. When an FLP recombinase is provided by a genetic cross with an FLP recombinase-expressing rodent, the ends of the first FRT and the second FRT are exchanged such that the reporter gene coding sequence is deleted, and the rodent Znrf3 cDNA coding sequence is rescued. When a Cre-recombinase is provided by a genetic cross with a Cre-expressing rodent, the rodent Znrf3 cDNA coding sequence is deleted resulting in the absence of rodent Znrf3 cDNA, and the rodent exhibits increased BMC and bone volume compared to a wild type rodent in which Znrf3 cDNA coding sequence is not deleted. In some embodiments, conditional knockout rodents having a genetic modification comprising at least one mutant Znrf3 allele that also comprises a PGK-Neo cassette located between the reporter gene coding sequence and the second FRT sequence, wherein when a FLP recombinase is provided via a genetic cross with a FLP recombinase-expressing rodent, the ends of the first FRT and the second FRT are exchanged such that the reporter gene coding sequence and PGK-Neo sequence are deleted and the Znrf3 coding sequence is rescued.

In some embodiments, the rodent Znrf3 cDNA coding sequence is a rat Znrf3 cDNA coding sequence. In some embodiments, the rat Znrf3 cDNA coding sequence comprises SEQ ID NO:2. In some embodiments, the rat Znrf3 cDNA coding sequence encodes a rat ZNRF3 protein comprising SEQ ID NO:4.

In some embodiments, the rodent Znrf3 cDNA coding sequence is a mouse Znrf3 cDNA coding sequence. In some embodiments, the mouse Znrf3 cDNA coding sequence comprises SEQ ID NO:1. In some embodiments, the mouse Znrf3 cDNA coding sequence encodes a mouse ZNRF3 protein comprising SEQ ID NO:3.

Similar to the knockouts described herein, insertion of a conditional knockout construct into an endogenous Znrf3 gene results in replacement of at least a portion of the endogenous Znrf3 gene sequence. In some embodiments, the rodent genomic fragment being replaced by a conditional knockout construct sequence comprises the nucleotide after the start codon through a subsequent coding exon from the endogenous rodent Znrf3 gene. In some embodiments, the rodent genomic fragment being replaced by a conditional knockout construct sequence comprises the ATG start codon (in the first coding exon) through the stop codon (in the last coding exon) of the endogenous rodent Znrf3 gene. In some embodiments, the rat Znrf3 cDNA coding sequence comprises the ATG start codon through the stop codon of the wild type rat Znrf3 gene. In some embodiments, the mouse Znrf3 cDNA coding sequence comprises the ATG start codon through the stop codon of the wild type mouse Znrf3 gene.

In some embodiments, the rodent genomic fragment being replaced by a conditional knockout construct sequence further comprises a 5′ non-coding exonic sequence or a 3′ non-coding exonic sequence of a rodent Znrf3 gene, or a combination thereof. In some embodiments, the genomic fragment being replaced by a conditional knockout construct sequence can also comprise the 5′ non-coding sequence in exon 1, or the 3′ UTR of exon 9 of the endogenous Znrf3 gene, or a combination thereof. In some embodiments, the rodent Znrf3 nucleic acid sequence in a conditional knockout rodent comprises a 5′ non-coding exonic sequence or a 3′ non-coding exonic sequence of a rodent Znrf3 gene, or a combination thereof. In embodiments, the rodent Znrf3 nucleic acid sequence in a conditional knockout Znrf3 comprises the 5′ non-coding sequence in exon 1, or the 3′ UTR of exon 9 of the rodent Znrf3 gene, or a combination thereof.

For the conditional knockout rodents described herein, any of the reporter gene coding sequences described herein can be used. The conditional knockout rodents described herein can be homozygous for the genetic modification. The conditional knockout rodents described herein can be heterozygous for the genetic modification.

In some embodiments, the conditional knockout rodents are rats. In some embodiments, the conditional knockout rodents are mice. In some embodiments, the rodent Znrf3 protein is from the same rodent species as the conditional knockout rodent (e.g., a mouse conditional knockout having a Znrf3 coding sequence that encodes mouse Znrf3). In some embodiments, the rodent Znrf3 protein is from a different rodent species as the conditional knockout rodent (e.g., a mouse conditional knockout having a Znrf3 coding sequence that encodes rat Znrf3).

The present disclosure also provides isolated rodent cells or tissue having a genetic modification comprising a disruption of one or both alleles of the Znrf3 gene. The isolated rodent cells or tissue can comprise any of the genetic modifications described herein for the knockout rodents and conditional knockout rodents. In some embodiments, the isolated rodent cells or tissue comprises an endogenous rodent Znrf3 gene that lacks a portion of the wild type rodent Znrf3 gene. In some embodiments, the isolated rodent cells or tissue comprises an endogenous rodent Znrf3 gene that lacks a fragment beginning from the nucleotide after the start codon through a subsequent coding exon from the wild type rodent Znrf3 gene. In some embodiments, the rodent genomic fragment being replaced comprises the ATG start codon (in the first coding exon) through the stop codon (in the last coding exon) of the endogenous rodent Znrf3 gene. In some embodiments, the rodent genomic fragment being replaced further comprises a 5′ non-coding exonic sequence or a 3′ non-coding exonic sequence of a rodent Znrf3 gene, or a combination thereof. In some embodiments, the rodent genomic fragment being replaced also comprises the 5′ non-coding sequence in exon 1, or the 3′ UTR of exon 9 of the endogenous Znrf3 gene, or a combination thereof. In some embodiments, the rodent genomic fragment being replaced comprises a fragment beginning from the nucleotide immediately after the start codon in the first coding exon through a subsequent coding exon (e.g., the second, third, fourth, fifth, sixth, seventh, eighth, or ninth coding exon).

In some embodiments, the isolated rodent cells or tissue comprise a genetically modified Znrf3 gene, wherein the genetic modification comprises a replacement of all or part of the endogenous rodent Znrf3 gene with a reporter gene, which forms part of the knockout construct. In some embodiments, the reporter gene is operably linked to the endogenous rodent Znrf3 promoter. In some embodiments, the reporter gene is inserted immediately downstream of the start codon (and is operably linked thereto) of the endogenous rodent Znrf3 gene. In such linkage, expression of the reporter gene is expected to resemble the expression pattern of an unmodified endogenous rodent Znrf3 gene.

In some embodiments, the isolated rodent cells or tissue have a genetic modification comprising at least one mutant Znrf3 allele that comprises, from 5′ to 3′, a first loxP site, a first FLP recombinase target (FRT) sequence, a reporter gene coding sequence, a second FRT sequence, a rodent Znrf3 cDNA coding sequence, and a second loxP site. In some embodiments, the isolated rodent cells or tissue have a genetic modification comprising at least one mutant Znrf3 allele that also comprises a PGK-Neo cassette located between the reporter gene coding sequence and the second FRT sequence.

In some embodiments, the rodent Znrf3 cDNA coding sequence is a rat Znrf3 cDNA coding sequence. In some embodiments, the rat Znrf3 cDNA coding sequence comprises SEQ ID NO:2. In some embodiments, the rat Znrf3 cDNA coding sequence encodes a rat ZNRF3 protein comprising SEQ ID NO:4. In some embodiments, the rodent Znrf3 cDNA coding sequence is a mouse Znrf3 cDNA coding sequence. In some embodiments, the mouse Znrf3 cDNA coding sequence comprises SEQ ID NO:1. In some embodiments, the mouse Znrf3 cDNA coding sequence encodes a mouse ZNRF3 protein comprising SEQ ID NO:3.

In some embodiments, the rodent genomic fragment being replaced by a conditional knockout construct sequence in the isolated rodent cells and tissue comprises the nucleotide after the start codon through a subsequent coding exon from the endogenous rodent Znrf3 gene. In some embodiments, the rodent genomic fragment being replaced by a conditional knockout construct sequence comprises the ATG start codon (in the first coding exon) through the stop codon (in the last coding exon) of the endogenous rodent Znrf3 gene. In some embodiments, the rat Znrf3 cDNA coding sequence comprises the ATG start codon through the stop codon of the wild type rat Znrf3 gene. In some embodiments, the mouse Znrf3 cDNA coding sequence comprises the ATG start codon through the stop codon of the wild type mouse Znrf3 gene.

In some embodiments, the rodent genomic fragment being replaced by a conditional knockout construct sequence in the isolated rodent cells and tissue further comprises a 5′ non-coding exonic sequence or a 3′ non-coding exonic sequence of a rodent Znrf3 gene, or a combination thereof. In some embodiments, the genomic fragment being replaced by a conditional knockout construct sequence can also comprise the 5′ non-coding sequence in exon 1, or the 3′ UTR of exon 9 of the endogenous Znrf3 gene, or a combination thereof. In some embodiments, the rodent Znrf3 nucleic acid sequence in the in the isolated rodent cells and tissue comprises a 5′ non-coding exonic sequence or a 3′ non-coding exonic sequence of a rodent Znrf3 gene, or a combination thereof. In embodiments, the rodent Znrf3 nucleic acid sequence in a conditional knockout Znrf3 comprises the 5′ non-coding sequence in exon 1, or the 3′ UTR of exon 9 of the rodent Znrf3 gene, or a combination thereof.

For the isolated rodent cells and tissue described herein, any of the reporter gene coding sequences described herein can be used. The isolated rodent cells and tissue described herein can be homozygous for the genetic modification. The isolated rodent cells and tissue described herein can be heterozygous for the genetic modification.

In some embodiments, the isolated rodent cells and tissue are rat cells and tissue. In some embodiments, the isolated rodent cells and tissue are mice cells and tissue.

The isolated rodent cells and tissue described herein include, but are not limited to, embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells; etc.; adipocytes, cells involved with particular organs, such as thymus, endocrine glands, pancreas, kidney, brain, such as neurons, glia, astrocytes, dendrocytes, hepatic cells, multipotent stem cells, lineage-committed stem cells, tumor cells, bone cells, chondrocytes, and chondrocyte precursors. In some embodiments, the isolated rodent cells or tissue are selected from the group consisting of a multipotent stem cell, a lineage-committed stem cell, a tumor cell, a chondrocyte, and a chondrocyte precursor.

In some embodiments the cells or tissues described herein are isolated from the genetically modified rodents described herein. Methods of isolating specific cell types are described in, for example, PCT Publications WO 1999/054439, WO 1996/007097, and WO 1999/011771.

The present disclosure also provides knockout DNA constructs for global or conditional inactivation of the rodent Znrf3 gene. Constructs include, but are not limited to, recombinant nucleic acid molecules, generally DNA, that have been generated for the purpose of the expression of a specific nucleotide sequence(s), or is to be used in the construction of other recombinant nucleotide sequences. Knockout constructs include, but are not limited to, nucleotide sequences that are designed to undergo homologous recombination with the endogenous targeted gene to disrupt it and thereby decrease or suppress expression of a polypeptide encoded by the targeted gene in one or more cells of a rodent. The nucleotide sequence used as the global knockout construct is typically comprised of: 1) a first DNA fragment from some portion of the targeted endogenous gene (which may include part or all of one or more exon sequences, intron sequences, and/or promoter sequences), 2) a selectable marker sequence used to detect the presence of the knockout construct in the cell and which serves as a basis for selecting cells carrying the disrupted recombined sequence, 3) optionally, a reporter gene which is only expressed in the event of a successful targeted recombinant event, and 4) a second DNA fragment from some portion of the targeted endogenous gene. The knockout constructs described herein can be inserted into a cell that comprises the endogenous rodent Znrf3 gene that is to be knocked out. The knockout construct can integrate with one or both alleles of the endogenous rodent Znrf3 gene, which results in the transcription of the full-length endogenous Znrf3 gene being disrupted or prevented. Integration of the Znrf3 knockout constructs described herein into the chromosomal DNA preferably takes place via homologous recombination, in which regions of the Znrf3 knockout construct exhibit a sufficient degree of homology with endogenous rodent Znrf3 genomic DNA sequences such that the construct, after insertion into a cell, can hybridize to the genomic DNA. This permits recombination between the construct and the genomic DNA leading to incorporation of the knockout construct into the corresponding position of the genomic DNA.

The present disclosure also provides knockout rodent Znrf3 DNA constructs comprising a selectable marker sequence or a reporter gene, or both, flanked by DNA sequences homologous to rodent Znrf3 genomic DNA. When the construct is introduced into an embryonic rodent cell, the selectable marker sequence disrupts the rodent Znrf3 gene in the embryonic cell, and the rodent resulting from the embryonic cell exhibits increased BMC and bone volume compared to a wild type rodent in which the Znrf3 gene is not disrupted.

In some embodiments, the knockout rodent Znrf3 DNA construct comprises, 5′ to 3′: a first rodent Znrf3 genomic DNA fragment; a selectable marker sequence and/or a reporter gene; and a second rodent Znrf3 genomic DNA fragment.

The selectable marker sequences are nucleotide sequences that are: 1) part of a larger knockout construct and is used to disrupt the expression of endogenous rodent Znrf3; and 2) used as a means to identify and to positively select those cells that have incorporated the Znrf3 knockout construct into the chromosomal DNA. The selectable marker sequence can be any sequence that serves these purposes, and typically encodes a protein that confers a detectable/selectable trait on the cell, such as an antibiotic resistance gene or an assayable enzyme not naturally found in the cell. The selectable marker sequence typically includes a homologous or heterologous promoter that drives expression of the marker. Suitable selectable marker sequences include, but are not limited to, neomycin phosphotransferase (neo), hygromycin B phosphotransferase, xanthine/guanine phosphoribosyl transferase, herpes simplex thymidine kinase (TK), and diphtheria toxin. In some embodiments, the selectable marker sequence comprises PGK-Neo cassette. PGK-Neo is a hybrid gene consisting of the phosphoglycerate kinase I promoter driving the neomycin phosphotransferase gene (resulting in neomycin resistance). This cassette can be employed as a selectable marker for homologous recombination in embryonic stem ES cells. In some embodiments, the selectable marker sequence is a neo cassette comprising a constitutive promoter.

The reporter gene can be any reporter gene described herein. In some embodiments, the reporter gene can encode a distinct surface antigen, a chromophore, a fluorescent protein, a chromogenic enzyme, or the like.

The present disclosure also provides vectors comprising any of the knockout rodent Znrf3 DNA constructs described herein.

In some embodiments, the knockout construct is a conditional knockout construct designed to maintain unaltered rodent Znrf3 expression in the primary targeted conformation and allow targeting of the rodent Znrf3 gene deletion exclusively to a particular organ or tissue, for example bone. In addition to regions of homology to the targeted genomic locus, a selectable marker sequence, and a reporter gene, such constructs generally include a “rescue gene” comprising a nucleic acid sequence that encodes the product of the gene being disrupted. This coding sequence may be operably linked to as promoter that is a part of insertion cassette of a conditional knockout construct, or it may become operably linked with the endogenous promoter upon incorporation at the target site in the genome. This allows for expression of the encoded gene product thereby “rescuing” the disruption. Thus, the cells where the genomic copy of the target (e.g., Znrf3) has been disrupted can still express the protein encoded by the targeted gene. This approach avoids the systemic problems (such as lack of viability or early death) that may arise in connection with global knockout of a gene.

The conditional knockout relies on the DNA recombination that allows excision of a portion of a knockout construct thereby activating the rescue gene or deleting the rescue gene sequence. Accordingly, in some embodiments, the conditional knockout constructs of further comprise one or more sets of recombination sites such as loxP or FRT sites which allow excision of all or part of sequences integrated into genomic target site. In some embodiments, the excision of the sequences flanked by loxP or FRT sites activates the rescue gene. In some embodiments, the excision of the sequences flanked by loxP or FRT sites removes the rescue gene sequences. In some embodiments, both a pair of loxP and a pair of FRT sites are present in the knockout construct, wherein the excision of the sequences flanked by one pair of recombination sites (for example FRT sites) activates the rescue gene, and excision of the sequences flanked by the other pair of recombination sites (for example loxP sites) removes the rescue gene sequences, resulting in no functional product being expressed from the disrupted gene locus. In some embodiments, the excision event that activates the rescue gene also removes other functional segments of the insertion cassette, such as the reporter gene sequences, the selectable marker gene sequences, or both. In some embodiments, the excision event that removes the rescue gene sequences also removes other functional segments of the insertion cassette, such as the reporter gene sequences, the marker gene sequences, or both.

Depending on size (e.g., whether a genomic DNA or cDNA is used), a rodent Znrf3 nucleic acid can be cloned directly from cDNA sources or synthetically made. Alternately, bacterial artificial chromosome (BAC) libraries can provide rodent Znrf3 nucleic acid sequences.

In some embodiments, the rescue gene encodes the exact protein as encoded by the gene being disrupted (i.e., from the same species). In some embodiments, the rescue gene encodes a protein from a different species that is homologous to the protein being disrupted. In some embodiments, the rescue gene is a wild type gene. In some embodiments, the rescue gene comprises one or more mutations.

In some embodiments, the conditional knockout rodent Znrf3 DNA construct comprises, in the 5′ to 3′ direction: a first rodent Znrf3 genomic DNA fragment; a first loxP site; a first FRT sequence; a reporter gene coding sequence; a second FRT sequence; a rodent Znrf3 cDNA coding sequence (i.e., a Znrf3 rescue gene coding sequence); a second loxP site; and a second rodent Znrf3 genomic DNA fragment. In some embodiments, the conditional knockout rodent Znrf3 DNA construct also comprises a selectable marker sequence, such as a PGK-Neo cassette, present between the reporter gene coding sequence and the second FRT sequence. In some embodiments, the conditional knockout rodent Znrf3 DNA construct also comprises a thymidine kinase cassette present between the second loxP site and the second rodent Znrf3 genomic DNA fragment. When an FLP-recombinase is provided (e.g., via a genetic cross with a FLP-expressing rodent), the ends of the first FRT and the ends of the second FRT are exchanged such that the reporter gene and the selectable marker sequence (if present) are deleted and the Znrf3 coding sequence is activated. When a Cre-recombinase is provided (e.g., via a genetic cross with a Cre-recombinase-expressing rodent), the Znrf3 cDNA coding sequence is deleted.

In some embodiments, the conditional knockout rodent Znrf3 DNA construct comprises, in the 5′ to 3′ direction: a first rodent Znrf3 genomic DNA fragment; a first FRT site; a first loxP sequence; a reporter gene coding sequence; a second loxP sequence; a rodent Znrf3 cDNA coding sequence (i.e., a Znrf3 rescue gene coding sequence); a second FRT site; and a second rodent Znrf3 genomic DNA fragment. In some embodiments, the conditional knockout rodent Znrf3 DNA construct also comprises a selectable marker sequence, such as a PGK-Neo cassette, present between the reporter gene coding sequence and the second loxP sequence. In some embodiments, the conditional knockout rodent Znrf3 DNA construct also comprises a thymidine kinase cassette present between the second FRT site and the second rodent Znrf3 genomic DNA fragment. When a Cre-recombinase is provided (e.g., via a genetic cross with a Cre-recombinase-expressing rodent), the ends of the first loxP and the second loxP are exchanged such that the reporter gene sequence and the selectable marker sequence (if present) is deleted and the Znrf3 cDNA coding sequence is activated. In some embodiments, when an FLP-recombinase is provided (e.g., via a genetic cross with a FLP-expressing rodent), the Znrf3 coding sequence is deleted resulting in the absence of Znrf3 cDNA at the disrupted Znrf3 gene locus.

In some embodiments, the rodent Znrf3 cDNA coding sequence is a rat Znrf3 cDNA coding sequence. In some embodiments, the rat Znrf3 cDNA coding sequence comprises SEQ ID NO:2. In some embodiments, the rat Znrf3 cDNA coding sequence encodes a rat ZNRF3 protein comprising SEQ ID NO:4. In some embodiments, the rodent Znrf3 cDNA coding sequence is a mouse Znrf3 cDNA coding sequence. In some embodiments, the mouse Znrf3 cDNA coding sequence comprises SEQ ID NO:1. In some embodiments, the mouse Znrf3 cDNA coding sequence encodes a mouse ZNRF3 protein comprising SEQ ID NO:3.

For the conditional knockout rodent Znrf3 DNA constructs, any of the reporter gene coding sequences described herein can be used.

The present disclosure also provides vectors comprising any of the conditional knockout rodent Znrf3 DNA constructs described herein.

The present disclosure methods of producing Znrf3 knockout rodents. Generally, the embryonic stem cells (ES cells) used to produce the knockout rodent are the same species as the knockout rodent to be generated. Thus, for example, mouse embryonic stem cells can be used to produce knockout mice. In some embodiments, the methods produce a genetically modified mouse. In some embodiments, the methods produce a genetically modified rat.

Embryonic stem cells are typically selected for their ability to integrate into and become part of the germ line of a developing embryo to create germ line transmission of the knockout construct. Thus, any ES cell line that is believed to have this capability is suitable for use herein. One mouse strain that is typically used for production of ES cells, is the 129J strain. A suitable ES cell line is the mouse cell line D3 (American Type Culture Collection Catalog No. CRL 1934). Other examples of suitable ES cell lines that can be used include, but are not limited to, mouse ES cell lines GS1-1 (previously BWE4) (Incyte Genomics, Inc. Palo Alto, Calif. USA) and R1 (Lunenfeld Research Institute, Mt. Sinai Hospital, Toronto, Ontario, Canada). As an alternative to ES cells, embryonic cells can be obtained freshly from a host, e.g. mouse, rat, guinea pig, etc.

ES or embryonic cells are typically grown on an appropriate fibroblast-feeder layer or in the presence of appropriate growth factors, such as leukemia inhibiting factor (LIF). When ES cells have been transformed, they are used to produce transgenic animals. After transformation, the cells are plated onto a feeder layer in an appropriate medium. Cells comprising the construct can be detected by employing a selective medium, such as medium with neomycin (or G418), when used in conjunction with the neo cassette. After sufficient time has passed for colonies to grow, colonies are picked and analyzed for the occurrence of homologous recombination/integration of the knockout construct. Those colonies that are positive may then be used for embryo manipulation and blastocyst injection. Representative methods used for cell culture and preparation for DNA insertion are described in, for example, Robertson, E. J., In: Teratocarcinomas and Embryonic Stein Cells: A Practical Approach, E. J. Robertson, ed. IRL Press, Washington, D.C. (1987); Bradley et al., Current Topics in Devel. Biol., 1986, 20, 357-371; Hogan et al., Manipulating the Mouse Embryo: A Laboratory Manual, Cold Spring Harbor Laboratory Press, Cold Spring Harbor, N.Y. (1986); and Talts et al., Meth. Mol. Biol., 1999, 129, 153-187.

The present disclosure provides methods of producing a genetically modified rodent, comprising: transforming a rodent embryonic stem cell with a knockout construct comprising a selectable marker sequence and/or a reporter gene sequence, flanked by DNA sequences homologous to the endogenous rodent Znrf3 genomic DNA, thereby producing a transformed embryonic stem cell; introducing the transformed embryonic stem cell into a rodent blastocyst; and implanting the blastocyst comprising the transformed embryonic stem cell into a pseudopregnant female rodent, and allowing the blastocyst to undergo fetal development to term, to produce the genetically modified rodent. The genetically modified rodent is a heterozygous knockout rodent and exhibits increased BMC and bone volume compared to a wild type rodent. In some embodiments, the genetically modified rodent is incapable of expressing an endogenous rodent ZNRF3 protein.

Insertion of the knockout construct into the ES cells can be accomplished using a variety of methods including, but not limited to, electroporation, microinjection, and calcium phosphate treatment. In some embodiments, the method of insertion is electroporation. In some embodiments, a targeting vector (such as a BAC vector) carrying any of the knockout constructs or conditional knockout constructs described herein, can be introduced into rodent embryonic stem (ES) cells by, for example, electroporation. Mouse ES cells, for example, can be transformed by methodologies such as VELOCIMOUSE®.

Each knockout construct DNA to be inserted into the cell is first linearized if the knockout construct has been inserted into a vector. Linearization can be accomplished by digesting the DNA with a suitable restriction endonuclease selected to cut only within the vector sequence and not within the knockout construct sequence.

For insertion of the DNA sequence, the knockout construct DNA can be added to the ES cells under appropriate conditions for the insertion method chosen. Where more than one construct is to be introduced into the ES cell, DNA encoding each construct can be introduced simultaneously or one at a time.

If the cells are to be electroporated, the ES cells and knockout construct DNA can be exposed to an electric pulse using an electroporation machine. After electroporation, the cells are allowed to recover under suitable incubation conditions. The cells are then screened for the presence of the knockout construct. Accordingly, in some embodiments, the methods further comprise testing the produced genetically modified rodent to verify that its genome comprises a disrupted Znrf3 gene in at least one allele.

Screening can be carried out using a variety of methods. If the selectable marker sequence is an antibiotic resistance gene, the cells can be cultured in the presence of an otherwise lethal concentration of antibiotic. Those cells that survive have presumably integrated the knockout construct. If the marker gene is other than an antibiotic resistance gene, a Southern blot of the ES cell genomic DNA can be probed with a sequence of DNA designed to hybridize only to the selectable marker sequence. If the selectable marker sequence is a gene that encodes an enzyme whose activity can be detected (e.g., S-galactosidase), the enzyme substrate can be added to the cells under suitable conditions, and the enzymatic activity can be analyzed.

The knockout construct can be integrated into several locations in the ES cell genome. In some embodiments, the desired location of the insertion is in a complementary position to the DNA sequence to be knocked out. Typically, less than about 1-5 percent of the ES cells that take up the knockout construct will actually integrate the knockout construct in the desired location. To identify those cells with proper integration of the knockout construct, the DNA can be extracted from the cells using standard methods such as those described by Sambrook et al., Molecular Cloning: A Laboratory Manual, 3rd Edition, Cold Spring Harbor Press, Cold Spring Harbor, N.Y., 2001; Brent et al., Current Protocols in Molecular Biology, John Wiley & Sons, Inc., 2003; and Ausubel et al., Short Protocols in Molecular Biology, 5th Edition, Current Protocols, 2002. The DNA can then be probed on a Southern blot with a probe or probes designed to hybridize in a specific pattern to genomic DNA digested with particular restriction enzyme(s). Alternately, or additionally, the genomic DNA can be amplified by PCR with probes specifically designed to amplify DNA fragments of a particular size and sequence (i.e., only those cells containing the knockout construct in the proper position will generate DNA fragments of the proper size).

After suitable ES cells containing the knockout construct in the proper location have been identified, the cells can be inserted into an embryo. Insertion can be carried out in a variety of ways, including microinjection. For microinjection, about 10-30 cells are collected into a micropipette and injected into embryos that are at the proper stage of development to integrate the ES cell into the developing embryo. The suitable stage of development for the embryo is very species dependent, but for mice it is about 3.5 days. The embryos can be obtained by perfusing the uterus of pregnant females. While any embryo of the right age/stage of development can be used, it may be preferable to use male embryos from strains of mice whose coat color is different from the coat color of the ES cell donor (or strain of origin). This facilitates screening for the presence of the knockout construct in mice with mosaic coat color (indicative of incorporation of the ES cell into the developing embryo).

The selected ES cells can be trypsinized and injected into a pre-morula stage embryo (e.g., 8-cell stage embryo) by using, for example, the VELOCIMOUSE® method (see, e.g., U.S. Pat. Nos. 7,576,259, 7,659,442, 7,294,754, and U.S. Patent Application Publication 2008/0078000), or methods described in, for example, U.S. Patent Application Publication 2014/0235933 and 2014/0310828. The embryo comprising the donor ES cells can be incubated until the blastocyst stage and then implanted into the uterine horns of pseudopregnant females. While any foster mother may be used, those preferred can be selected for their past breeding ability and tendency to care well for their young. Suitable foster mothers are used when about 2-3 days pseudo-pregnant. Pregnancies are allowed to proceed to term and birth of pups. The resulting litters are screened for mutant cells comprising the construct.

Offspring (progeny) that are born to the foster mother can be screened initially for mosaic coat color where the coat color selection strategy (as described herein) has been employed. In addition, or as an alternative, DNA from tail tissue of the offspring can be screened for the presence of the knockout construct using Southern blots and/or PCR as described herein. Offspring that appear to be mosaics can be crossed to each other if they are believed to carry the knockout construct in their germ line to generate homozygous knockout rodents. If it is unclear whether the offspring will have germ line transmission, they can be crossed with a parental or other strain and the offspring screened for heterozygosity. The heterozygotes can be identified by Southern blots and/or PCR amplification of the DNA, as set forth herein.

Other means of identifying and characterizing the knockout offspring are available. For example, Northern blots can be used to probe the mRNA from the rodent for the presence or absence of transcripts encoding either the gene knocked out, the selectable marker sequence, or both. Western blots can be used to assess the level of expression of the knocked out gene in various tissues of these offspring by probing with an antibody against the ZNRF3 protein. In situ analysis, such as fixing tissue or blood cells from the knockout rodent, and labelling with antibody and/or flow cytometric analysis of various cells from the offspring can also be conducted. This method works well with suitable anti-ZNRF3 antibodies.

Further provided herein are methods of breeding a genetically modified rodent as described herein with another rodent, as well as progenies obtained from such breeding. In some embodiments, the methods comprise breeding a first rodent whose genome comprises a disrupted rodent Znrf3 gene with a second rodent, resulting in a progeny rodent whose genome comprises the a disrupted Znrf3 gene. In some embodiments, the methods of producing a Znrf3 knockout rodent having a genome which is homozygous for a disruption of the rodent Znrf3 gene comprise: breeding a first heterozygous knockout rodent produced in accordance with any one of claims 61 to 65 with a second heterozygous knockout rodent to produce a progeny rodent; and selecting a progeny rodent in which the disruption of the Znrf3 gene is homozygous. In some embodiments, the produced rodent is incapable of expressing an endogenous rodent ZNRF3 protein. In some embodiments, the first heterozygous knockout rodent, second heterozygous knockout rodent, and progeny rodent are mice. In some embodiments, the first heterozygous knockout rodent, second heterozygous knockout rodent, and progeny rodent are rats. The present disclosure also provides progeny rodents produced by these methods.

Progeny include any and all future generations of rodents derived or descendant from a particular progenitor rodent, such as a KO rodent, such as a KO mouse or rat in which the endogenous Znrf3 gene has been disrupted (whether heterozygous or homozygous for the disruption). Progeny of any successive generation are included herein such that the progeny, the F1, F2, F3, generations and so on indefinitely, comprising the disrupted gene (with the knockout construct) are included.

In some embodiments, methods are provided which comprise breeding a first genetically modified rodent as described herein (e.g., a rodent whose genome comprises a disrupted Znrf3 gene), with a second rodent resulting in a progeny rodent whose genome comprises the disrupted Znrf3 gene. The progeny can possess other desirable phenotypes or genetic modifications inherited from the second rodent used in the breeding. In some embodiments, the progeny rodent is heterozygous for the disrupted Znrf3 gene. In some embodiments, the progeny rodent is homozygous for the disrupted Znrf3 gene. In some embodiments, the first rodent and the second rodent are mice. In some embodiments, the first rodent and the second rodent are rats.

Homozygotes can be identified by Southern blotting of equivalent amounts of genomic DNA from mice that are the product of this cross, as well as mice that are known heterozygotes and wild type mice. Probes to screen the Southern blots can be designed as set forth herein.

Other means of identifying and characterizing the knockout offspring are available. For example, Northern blots can be used to probe the mRNA for the presence or absence of transcripts encoding either the gene knocked out, the selectable marker sequence, or both. In addition, Western blots can be used to assess the level of expression of the gene knocked out in various tissues of these offspring by probing the Western blot with an antibody against the protein (ZNRF3) encoded by the gene knocked out, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

The knockout rodents described herein, and cells and tissue obtained therefrom, have a variety of uses described herein. One use of the KO rodents and their progeny is as a model for development of bone mineral density or BMC. Accordingly, the present disclosure provides rodent models of increased bone mineral density comprising any of the genetically modified rodents described herein whose genome comprises any of the genetically modified Znrf3 genes described herein. The rodent can be heterozygous or homozygous for the genetic modification. The KO rodents described herein can also be used, for example, to investigate chemical pathways involved in bone mineral density or BMC and to further investigate the role of the Znrf3 gene and gene product in bone mineral density and/or BMC. In some embodiments, wild type ZNRF3 protein can be administered to any of the rodents within the rodent models described herein. In such methods, reversion to wild type phenotype regarding decreased bone mineral density and/or BMC can be examined.

The present KO rodents can be used to screen an agent(s) for activity in preventing, inhibiting, alleviating, or reversing symptoms associated with increased bone mineral density and/or BMC. Such an agent may be a chemical compound, a drug, a macromolecule such as a nucleic acid (DNA, RNA, PNA), a polypeptide or fragments thereof; an antibody or fragments thereof; a peptide, such as an oligopeptide; or a mixture of any of the above. Also, the agent may be a mixture of agents obtained from natural sources, such as microorganisms, plants or animals. In some embodiments, the agent is wild type ZNRF3 protein.

Screening a series of agents for their activity as a potentially useful drug involves administering the agent over a range of doses to the Znrf3 KO mice, and evaluating the status of the mice with respect to increased bone mineral density and/or BMC. In some embodiments, the methods of determining the effect of an agent for treating high bone mineral density and/or BMC comprise: administering the agent to a rodent that is heterozygous or homozygous for a Znrf3 gene knockout; subjecting the rodent to a test to assess bone mineral density and/or BMC; and determining whether the agent has any effect on the bone mineral density and/or BMC in the rodent.

Agents can be screened for their ability to mitigate an undesirable phenotype (e.g., a symptom) associated with absent or reduced Znrf3 expression or function. In some embodiments, screening of candidate agents is performed in vivo in any of the KO rodents described herein. In some embodiments, the candidate agent is administered to the Znrf3 KO rodent and the effects of the candidate agent are determined. The candidate agent can be administered in any manner desired and/or appropriate for delivery of the agent in order to affect a desired result. For example, the candidate agent can be administered by injection or infusion, e.g., intravenously, intramuscularly, subcutaneously, or directly into the tissue in which the desired affect is to be achieved, orally, or by any other desired route. In some embodiments, the in vivo screen will involve a number of animals receiving varying amounts and concentrations of the candidate agent (ranging from negative controls to an amount of agent that approaches an upper limit of tolerable doses) and may include delivery of the agent in any of a number of different formulations. The agents can be administered singly or can be in combinations of two or more agents, especially where administration of a combination of agents may result in a synergistic effect. The effect of the test agent upon the KO rodent can be monitored by assessing a biological function as appropriate or by assessing a phenotype associated with the loss of Znrf3 function. The effect of the candidate agent can be assessed by Dual Energy Xray Absorptiometry (DEXA), Quantitative computed tomography (QCT), single energy absorptiometry, metacarpal width or density from hand xrays, magnetic resonance imaging, or ultrasound densitometer test, and determining whether the candidate therapeutic agent has any effect on the performance of the rodent in the test, e.g., any improvement of bone mineralization. In some embodiments, the test is a DEXA test. In some embodiments, the test is a QCT test. Where the candidate agent affects a Znrf3-associated phenotype in a desired manner, the candidate agent is identified as an agent suitable for use in therapy of a Znrf3-associated disorder.

Therapeutic agents that can be tested in any of the KO rodents described herein include both commercially available agents and candidate compounds under development for treating decreased bone mineral density. Both small molecule chemical compounds and nucleic acids (e.g., gene therapy drugs) can be tested. Znrf3-associated disorders for which an agent suitable for use in therapy is identified include, but are not limited to, osteoarthritic spondylosis, diffuse idiopathic skeletal hyperostosis (DISH), ankylosing spondylotis, Paget's disease, tuberous sclerosis, osteosclerosis, renal osteodystrophy, synovitis/acne/pustulosis/hyperostosis/osteitis (SAPHO) syndrome, acromegaly, Hepatitis C-associated osteosclerosis, myelofibrosis, and mastocytosis.

Administration of a therapeutic agent to a KO rodent can be by any suitable route including, but not limited to, parenteral, intravenous, oral, subcutaneous, intra-arterial, intracranial, intrathecal, intraperitoneal, topical, intranasal, or intramuscular. Administration can also be by continuous infusion, local administration, sustained release from implants (gels, membranes or the like), and/or intravenous injection.

In order that the subject matter disclosed herein may be more efficiently understood, examples are provided below. It should be understood that these examples are for illustrative purposes only and are not to be construed as limiting the claimed subject matter in any manner.

The following representative embodiments are presented:

Embodiment 1. A genetically modified rodent having a genetic modification comprising a disruption of one or both alleles of the Zinc and Ring Finger 3 (Znrf3) gene, wherein when at least one allele is disrupted, the rodent exhibits increased bone mineral content (BMC) and bone volume compared to a wild type rodent in which neither Znrf3 allele is disrupted.

Embodiment 2. The genetically modified rodent according to embodiment 1, wherein an endogenous rodent Znrf3 gene lacks a portion of the wild type rodent Znrf3 gene.

Embodiment 3. The genetically modified rodent according to embodiment 1 or embodiment 2, wherein the endogenous rodent Znrf3 gene lacks a fragment beginning from the nucleotide after the start codon through a subsequent coding exon from the wild type rodent Znrf3 gene.

Embodiment 4. The genetically modified rodent according to any one of embodiments 1 to 3, wherein the endogenous rodent Znrf3 promoter is operably linked to a reporter gene.

Embodiment 5. The genetically modified rodent according to embodiment 4, wherein the reporter gene is operably linked to the start codon of the endogenous rodent Znrf3 gene.

Embodiment 6. The genetically modified rodent according to embodiment 4 or embodiment 5, wherein the reporter gene is LacZ, or a gene encoding a protein selected from the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP). Discosoma sp. red fluorescent protein (DsRed), and Meandrina meandrites green fluorescent protein (MmGFP).

Embodiment 7. The genetically modified rodent according to any one of embodiments 1 to 6, wherein the rodent is homozygous for the genetic modification.

Embodiment 8. The genetically modified rodent according to any one of embodiments 1 to 6, wherein the rodent is heterozygous for the genetic modification.

Embodiment 9. The genetically modified rodent according to any one of embodiments 1 to 8, wherein the rodent is a rat.

Embodiment 10. The genetically modified rodent according to any one of embodiments 1 to 8, wherein the rodent is a mouse.

Embodiment 11. A conditional knockout rodent having a genetic modification comprising at least one mutant Znrf3 allele that comprises, from 5′ to 3′, a first loxP site, a first FLP recombinase target (FRT) sequence, a reporter gene coding sequence, a second FRT sequence, a rodent Znrf3 cDNA coding sequence, and a second loxP site, wherein: when an FLP recombinase is provided by a genetic cross with an FLP recombinase-expressing rodent, the ends of the first FRT and the second FRT are exchanged such that the reporter gene coding sequence is deleted, and the rodent Znrf3 cDNA coding sequence is rescued; and when a Cre-recombinase is provided by a genetic cross with a Cre-expressing rodent, the rodent Znrf3 cDNA coding sequence is deleted resulting in the absence of rodent Znrf3 cDNA, and the rodent exhibits increased bone mineral content (BMC) and bone volume compared to a wild type rodent in which Znrf3 cDNA coding sequence is not deleted.

Embodiment 12. The conditional knockout rodent according to embodiment 11, wherein the rodent Znrf3 cDNA coding sequence is a rat Znrf3 cDNA coding sequence.

Embodiment 13. The conditional knockout rodent according to embodiment 12, wherein the rat Znrf3 cDNA coding sequence comprises the ATG start codon through the stop codon of the wild type rat Znrf3 gene.

Embodiment 14. The conditional knockout rodent according to embodiment 13, wherein the rat Znrf3 cDNA coding sequence comprises SEQ ID NO:2.

Embodiment 15. The conditional knockout rodent according to embodiment 11, wherein the rodent Znrf3 cDNA coding sequence is a mouse Znrf3 cDNA coding sequence.

Embodiment 16. The conditional knockout rodent according to embodiment 15, wherein the mouse Znrf3 cDNA coding sequence comprises the ATG start codon through the stop codon of the wild type mouse Znrf3 gene.

Embodiment 17. The conditional knockout rodent according to embodiment 16, wherein the mouse Znrf3 cDNA coding sequence comprises SEQ ID NO:1.

Embodiment 18. The conditional knockout rodent according to any one of embodiments 11 to 17, wherein the reporter gene coding sequence encodes LacZ, or encodes a protein selected from the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), Discosoma sp. red fluorescent protein (DsRed), and Meandrina meandrites green fluorescent protein (MmGFP).

Embodiment 19. The conditional knockout rodent according to any one of embodiments 11 to 18, wherein the rodent is homozygous for the genetic modification.

Embodiment 20. The conditional knockout rodent according to any one of embodiments 11 to 18, wherein the rodent is heterozygous for the genetic modification.

Embodiment 21. The conditional knockout rodent according to any one of embodiments 11 to 20, wherein the rodent is a rat.

Embodiment 22. The conditional knockout rodent according to any one of embodiments 11 to 20, wherein the rodent is a mouse.

Embodiment 23. An isolated rodent cell or tissue having a genetic modification comprising a disruption of one or both alleles of the Zinc and Ring Finger 3 (Znrf3) gene.

Embodiment 24. The isolated rodent cell or tissue according to embodiment 23, wherein an endogenous rodent Znrf3 gene lacks a portion of the wild type rodent Znrf3 gene.

Embodiment 25. The isolated rodent cell or tissue according to embodiment 23 or embodiment 24, wherein the endogenous rodent Znrf3 gene lacks a fragment beginning from the nucleotide after the start codon through a subsequent coding exon from the wild type rodent Znrf3 gene.

Embodiment 26. The isolated rodent cell or tissue according to any one of embodiments 23 to 25, wherein the endogenous rodent Znrf3 promoter is operably linked to a reporter gene.

Embodiment 27. The isolated rodent cell or tissue according to embodiment 26, wherein the reporter gene is operably linked to the start codon of the endogenous rodent Znrf3 gene.

Embodiment 28. The isolated rodent cell or tissue according to embodiment 26 or embodiment 27, wherein the reporter gene is LacZ, or a gene encoding a protein selected from the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), Discosoma sp. red fluorescent protein (DsRed), and Meandrina meandrites green fluorescent protein (MmGFP).

Embodiment 29. The isolated rodent cell or tissue according to any one of embodiments 23 to 28, wherein the isolated rodent cell or tissue is homozygous for the genetic modification.

Embodiment 30. The isolated rodent cell or tissue according to any one of embodiments 23 to 29, wherein the isolated rodent cell or tissue is heterozygous for the genetic modification.

Embodiment 31. The isolated rodent cell or tissue according to any one of embodiments 23 to 30, wherein the isolated rodent cell or tissue is a rat cell or tissue.

Embodiment 32. The isolated rodent cell or tissue according to any one of embodiments 23 to 30, wherein the isolated rodent cell or tissue is a mouse cell or tissue.

Embodiment 33. The isolated rodent cell or tissue according to any one of embodiments 23 to 32, wherein the cell or tissue is selected from the group consisting of a multipotent stem cell, a lineage-committed stem cell, a tumor cell, a bone cell, a chondrocyte, and a chondrocyte precursor.

Embodiment 34. An isolated rodent cell or tissue having a genetic modification comprising at least one mutant Znrf3 allele that comprises, from 5′ to 3′, a first loxP site, a first FLIP recombinase target (FRT) sequence, a reporter gene coding sequence, a second FRT sequence, a rodent Znrf3 cDNA coding sequence, and a second loxP site, wherein: when an FLP recombinase is provided by a genetic cross with an FLIP recombinase-expressing rodent, the ends of the first FRT and the second FRT are exchanged such that the reporter gene coding sequence is deleted, and the rodent Znrf3 cDNA coding sequence is rescued; and when a Cre-recombinase is provided by a genetic cross with a Cre-expressing rodent, the rodent Znrf3 cDNA coding sequence is deleted resulting in the absence of rodent Znrf3 cDNA, and the rodent exhibits increased bone mineral content (BMC) and bone volume compared to a wild type rodent in which Znrf3 cDNA coding sequence is not deleted.

Embodiment 35. The isolated rodent cell or tissue according to embodiment 34, wherein the rodent Znrf3 cDNA coding sequence is a rat Znrf3 cDNA coding sequence.

Embodiment 36. The isolated rodent cell or tissue according to embodiment 35, wherein the rat Znrf3 cDNA coding sequence comprises the ATG start codon through the stop codon of the wild type rat Znrf3 gene.

Embodiment 37. The isolated rodent cell or tissue according to embodiment 36, wherein the rat Znrf3 cDNA coding sequence comprises SEQ ID NO:2.

Embodiment 38. The isolated rodent cell or tissue according to embodiment 34, wherein the rodent Znrf3 cDNA coding sequence is a mouse Znrf3 cDNA coding sequence.

Embodiment 39. The isolated rodent cell or tissue according to embodiment 38, wherein the mouse Znrf3 cDNA coding sequence comprises the ATG start codon through the stop codon of the wild type mouse Znrf3 gene.

Embodiment 40. The isolated rodent cell or tissue according to embodiment 39, wherein the mouse Znrf3 cDNA coding sequence comprises SEQ ID NO:1.

Embodiment 41. The isolated rodent cell or tissue according to any one of embodiments 34 to 40, wherein the reporter gene coding sequence encodes LacZ, or encodes a protein selected from the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), Discosoma sp. red fluorescent protein (DsRed), and Meandrina meandrites green fluorescent protein (MmGFP).

Embodiment 42. The isolated rodent cell or tissue according to any one of embodiments 34 to 41, wherein the rodent cell or tissue is homozygous for the genetic modification.

Embodiment 43. The isolated rodent cell or tissue according to any one of embodiments 34 to 41, wherein the rodent cell or tissue is heterozygous for the genetic modification.

Embodiment 44. The isolated rodent cell or tissue according to any one of embodiments 34 to 43, wherein the rodent cell or tissue is a rat cell or tissue.

Embodiment 45. The isolated rodent cell or tissue according to any one of embodiments 34 to 43, wherein the rodent cell or tissue is a mouse cell or tissue.

Embodiment 46. The isolated rodent cell or tissue according to any one of embodiments 34 to 45, wherein the cell or tissue is selected from the group consisting of a multipotent stem cell, a lineage-committed stem cell, a tumor cell, a bone cell, a chondrocyte, and a chondrocyte precursor.

Embodiment 47. A knockout rodent Znrf3 DNA construct comprising a selectable marker sequence or a reporter gene, or both, flanked by DNA sequences homologous to rodent Znrf3 genomic DNA, wherein when the construct is introduced into an embryonic rodent cell, the selectable marker sequence disrupts the rodent Znrf3 gene in the embryonic cell, and the rodent resulting from the embryonic cell exhibits increased bone mineral content (BMC) and bone volume compared to a wild type rodent in which the Znrf3 gene is not disrupted.

Embodiment 48. The knockout rodent Znrf3 DNA construct according to embodiment 47, wherein the construct comprises, 5′ to 3′: a first rodent Znrf3 genomic DNA fragment; a selectable marker sequence and/or a reporter gene; and a second rodent Znrf3 genomic DNA fragment.

Embodiment 49. The knockout rodent Znrf3 DNA construct according to embodiment 48, wherein the reporter gene is LacZ, or a gene encoding a protein selected the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, and MmGFP.

Embodiment 50. The knockout rodent Znrf3 DNA construct according to embodiment 48 or embodiment 49, wherein the selectable marker sequence is a neo cassette comprising a constitutive promoter.

Embodiment 51. A vector comprising the knockout rodent Znrf3 DNA construct according to any one of embodiments 47 to 50.

Embodiment 52. A conditional knockout rodent Znrf3 DNA construct comprising, in the 5′ to 3′ direction: a first rodent Znrf3 genomic DNA fragment; a first loxP site; a first FRT sequence; a reporter gene coding sequence; a second FRT sequence; a rodent Znrf3 cDNA coding sequence; a second loxP site; and a second rodent Znrf3 genomic DNA fragment.

Embodiment 53. The conditional knockout rodent Znrf3 DNA construct according to embodiment 52, wherein the rodent Znrf3 cDNA coding sequence is a rat Znrf3 cDNA coding sequence.

Embodiment 54. The conditional knockout rodent Znrf3 DNA construct according to embodiment 53, wherein the rat Znrf3 cDNA coding sequence comprises the ATG start codon through the stop codon of the wild type rat Znrf3 gene.

Embodiment 55. The conditional knockout rodent Znrf3 DNA construct according to embodiment 54, wherein the rat Znrf3 cDNA coding sequence comprises SEQ ID NO:2.

Embodiment 56. The conditional knockout rodent Znrf3 DNA construct according to embodiment 52, wherein the rodent Znrf3 cDNA coding sequence is a mouse Znrf3 cDNA coding sequence.

Embodiment 57. The conditional knockout rodent Znrf3 DNA construct according to embodiment 56, wherein the mouse Znrf3 cDNA coding sequence comprises the ATG start codon through the stop codon of the wild type mouse Znrf3 gene.

Embodiment 58. The conditional knockout rodent Znrf3 DNA construct according to embodiment 57, wherein the mouse Znrf3 cDNA coding sequence comprises SEQ ID NO:1.

Embodiment 59. The conditional knockout rodent Znrf3 DNA construct according to any one of embodiments 52 to 58, wherein the reporter gene coding sequence encodes LacZ, or encodes a protein selected from the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, and MmGFP.

Embodiment 60. A vector comprising the conditional knockout rodent Znrf3 DNA construct according to any one of the embodiments 52 to 59.

Embodiment 61. A method of producing a genetically modified rodent, comprising: transforming a rodent embryonic stem cell with a knockout construct comprising a selectable marker sequence and/or a reporter gene sequence, flanked by DNA sequences homologous to the endogenous rodent Znrf3 genomic DNA, thereby producing a transformed embryonic stem cell; introducing the transformed embryonic stem cell into a rodent blastocyst; and implanting the blastocyst comprising the transformed embryonic stem cell into a pseudopregnant female rodent, and allowing the blastocyst to undergo fetal development to term, to produce the genetically modified rodent; wherein the genetically modified rodent is a heterozygous knockout rodent and exhibits increased bone mineral content (BMC) and bone volume compared to a wild type rodent.

Embodiment 62. The method according to embodiment 61, further comprising testing the produced genetically modified rodent to verify that its genome comprises a disrupted Znrf3 gene in at least one allele.

Embodiment 63. The method according to embodiment 61 or embodiment 62, wherein the genetically modified rodent is incapable of expressing an endogenous rodent ZNRF3 protein.

Embodiment 64. The method according to any one of embodiments 61 to 63, wherein the genetically modified rodent is a mouse.

Embodiment 65. The method according to any one of embodiments 61 to 63, wherein the genetically modified rodent is a rat.

Embodiment 66. The method according to any one of embodiments 61 to 65, wherein the reporter gene is LacZ, or a gene encoding a protein selected the group consisting of luciferase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, and MmGFP.

Embodiment 67. A method of producing a Znrf3 knockout rodent having a genome which is homozygous for a disruption of the rodent Znrf3 gene, the method comprising: breeding a first heterozygous knockout rodent produced in accordance with any one of embodiments 61 to 65 with a second heterozygous knockout rodent to produce a progeny rodent; and selecting a progeny rodent in which the disruption of the Znrf3 gene is homozygous.

Embodiment 68. The method according to embodiment 67, wherein the rodent is incapable of expressing an endogenous rodent ZNRF3 protein.

Embodiment 69. The method according to embodiment 67 or embodiment 68, wherein the first heterozygous knockout rodent, second heterozygous knockout rodent, and progeny rodent are mice.

Embodiment 70. The method according to embodiment 67 or embodiment 68, wherein the first heterozygous knockout rodent, second heterozygous knockout rodent, and progeny rodent are rats.

Embodiment 71. A progeny rodent produced by the method of any one of embodiments 61 to 70.

Embodiment 72. A method of determining the effect of an agent for treating high bone mineral density and/or bone mineral content, the method comprising: administering the agent to a rodent that is heterozygous or homozygous for a Znrf3 gene knockout; subjecting the rodent to a test to assess bone mineral density and/or bone mineral content; and determining whether the agent has any effect on the bone mineral density and/or bone mineral content in the rodent.

Embodiment 73. The method according to embodiment 72, wherein the test is a Dual Energy Xray Absorptiometry (DEXA) test.

Embodiment 74. The method according to embodiment 72, wherein the test is Quantitative Computed Tomography (QCT) test.

Embodiment 75. A rodent model of increased bone mineral density and/or bone mineral content, wherein the rodent is heterozygous or homozygous for a Znrf3 gene knockout.

Embodiment 76. The rodent model according to embodiment 75, wherein the rodent is a rodent according to any one of embodiments 1 to 22.

### EXAMPLES

### Example 1: Generation of Genetically Modified Mice

Mice deficient in Znrf3 were generated by homologous recombination using the VELOCIGENE® technology (Valenzuela et al., Nat. Biotechnol., 2003, 21, 652-659; and Poueymirou, Nat. Biotech., 2007, PMID:17187059) in which a 4134 bp segment (SEQ ID NO:5) containing the very start of cEx5 to the very end of cEx7 was deleted and replaced with LacZ fused in-frame.

In particular, ZNRF3 targeting constructs were designed as follows. For knockout of mouse ZNRF3, a cloning vector containing a synthesized 1 kb upstream arm and 1 kb downstream arm of deleted 4134 bp mouse ZNRF3 genomic sequence was constructed such that a floxed (i.e., flanking by loxP) TM lacZ reporter cassette containing a neomycin resistance gene under the control of the human UBC (ubiquitin) promoter replaced most of ZNRF3 exon 5 (just after the first 6 bp nucleotide sequence of CAACCC (amino acid residue 209-210) to the very end of exon 7 (6 bp away from the end)). The cassette was cloned such that the lacZ coding sequence was in frame. This construct was electroporated into 100% C57Bl/6NTac mouse embryonic stem cells. These constructs were electroporated into a 50% C57Bl/6NTac/50% 129SvEvTac mouse embryonic stem cell line. Successfully targeted clones from all electroporations were identified by TAQMAN analysis. ZNRF3^{-/+} and ZNRF3^{-/-} mice were generated using the VELOCIGENE® method and backcrossed to C57Bl/6NTac.

### Example 2: Assessing Expression of ZNRF3 Through a Reporter

Using genetically modified mice which comprise a deletion in an endogenous mouse ZNRF3 gene and an insertion of a reporter gene, wherein the reporter gene is operably linked to the endogenous mouse ZNRF3 promoter at the endogenous mouse ZNRF3 locus, the expression of ZNRF3 in adult mice was confirmed.

### Example 3: Assessing BMD in ZNRF3 Knockout Mice

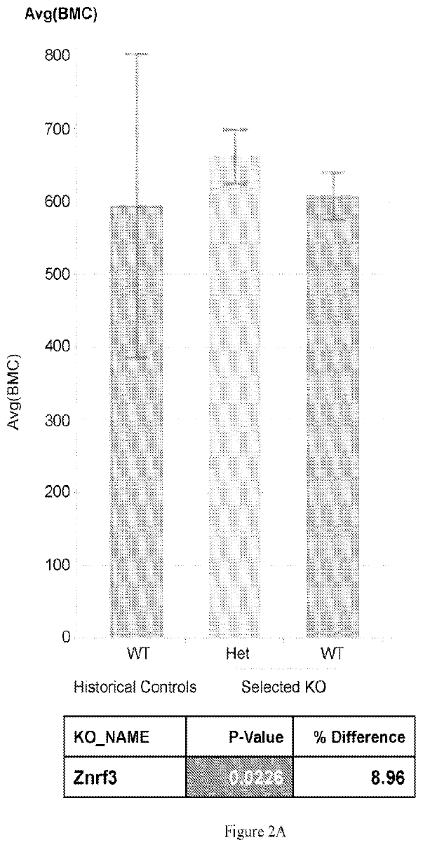

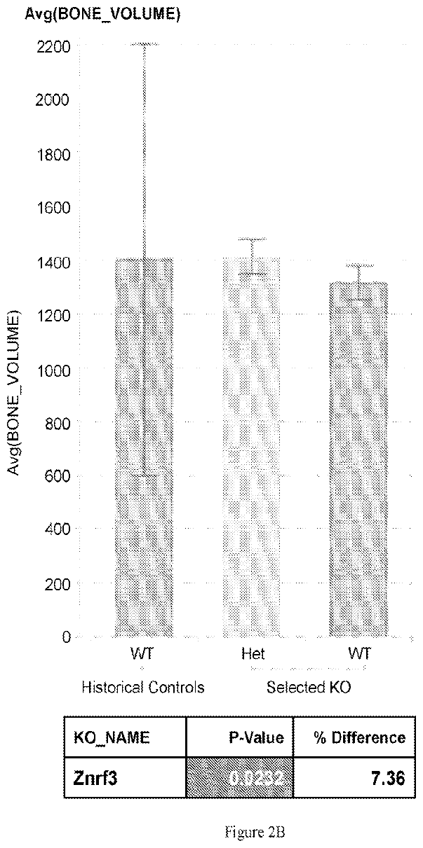

Bone mineral content (BMC) and bone volume was assessed in heterozygous Znrf3 null mice. The results show that heterozygous Znrf3 null mice have increased BMC (p=0.02, % difference=8.96) and increased bone volume (p=0.02, % difference=7.36) compared to their wildtype littermates (see, FIGS. 2, 3A, and 38).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A genetically modified rodent having a genetic modification comprising a disruption of one or both alleles of the Zinc and Ring Finger 3 (Znrf3) gene, wherein when at least one allele is disrupted, the rodent exhibits increased bone mineral content (BMC) and bone volume compared to a wild type rodent in which neither Znrf3 allele is disrupted.

2. The genetically modified rodent according to claim 1, wherein an endogenous rodent Znrf3 gene lacks a portion of the wild type rodent Znrf3 gene.

3. The genetically modified rodent according to claim 1 or claim 2, wherein the endogenous rodent Znrf3 promoter is operably linked to a reporter gene.

4. A conditional knockout rodent having a genetic modification comprising at least one mutant Znrf3 allele that comprises, from 5′ to 3′, a first loxP site, a first FLP recombinase target (FRT) sequence, a reporter gene coding sequence, a second FRT sequence, a rodent Znrf3 cDNA coding sequence, and a second loxP site, wherein:
when an FLP recombinase is provided by a genetic cross with an FLP recombinase-expressing rodent, the ends of the first FRT and the second FRT are exchanged such that the reporter gene coding sequence is deleted, and the rodent Znrf3 cDNA coding sequence is rescued; and
when a Cre-recombinase is provided by a genetic cross with a Cre-expressing rodent, the rodent Znrf3 cDNA coding sequence is deleted resulting in the absence of rodent Znrf3 cDNA, and the rodent exhibits increased bone mineral content (BMC) and bone volume compared to a wild type rodent in which Znrf3 cDNA coding sequence is not deleted.

5. The conditional knockout rodent according to claim 4, wherein the rodent Znrf3 cDNA coding sequence is a rat Znrf3 cDNA coding sequence.

6. The conditional knockout rodent according to claim 5, wherein the rat Znrf3 cDNA coding sequence comprises the ATG start codon through the stop codon of the wild type rat Znrf3 gene.

7. The conditional knockout rodent according to claim 4, wherein the rodent Znrf3 cDNA coding sequence is a mouse Znrf3 cDNA coding sequence.

8. The conditional knockout rodent according to claim 7, wherein the mouse Znrf3 cDNA coding sequence comprises the ATG start codon through the stop codon of the wild type mouse Znrf3 gene.

9. An isolated rodent cell or tissue having a genetic modification comprising a disruption of one or both alleles of the Zinc and Ring Finger 3 (Znrf3) gene.

10. The isolated rodent cell or tissue according to claim 9, wherein an endogenous rodent Znrf3 gene lacks a portion of the wild type rodent Znrf3 gene.

11. The isolated rodent cell or tissue according to claim 9 or claim 10, wherein the endogenous rodent Znrf3 promoter is operably linked to a reporter gene.

12. An isolated rodent cell or tissue having a genetic modification comprising at least one mutant Znrf3 allele that comprises, from 5′ to 3′, a first loxP site, a first FLIP recombinase target (FRT) sequence, a reporter gene coding sequence, a second FRT sequence, a rodent Znrf3 cDNA coding sequence, and a second loxP site, wherein:
when an FLP recombinase is provided by a genetic cross with an FLIP recombinase-expressing rodent, the ends of the first FRT and the second FRT are exchanged such that the reporter gene coding sequence is deleted, and the rodent Znrf3 cDNA coding sequence is rescued; and
when a Cre-recombinase is provided by a genetic cross with a Cre-expressing rodent, the rodent Znrf3 cDNA coding sequence is deleted resulting in the absence of rodent Znrf3 cDNA, and the rodent exhibits increased bone mineral content (BMC) and bone volume compared to a wild type rodent in which Znrf3 cDNA coding sequence is not deleted.

13. The isolated rodent cell or tissue according to claim 12, wherein the rodent Znrf3 cDNA coding sequence is a rat Znrf3 cDNA coding sequence.

14. The isolated rodent cell or tissue according to claim 13, wherein the rat Znrf3 cDNA coding sequence comprises the ATG start codon through the stop codon of the wild type rat Znrf3 gene.

15. The isolated rodent cell or tissue according to claim 12, wherein the rodent Znrf3 cDNA coding sequence is a mouse Znrf3 cDNA coding sequence.

16. The isolated rodent cell or tissue according to claim 15, wherein the mouse Znrf3 cDNA coding sequence comprises the ATG start codon through the stop codon of the wild type mouse Znrf3 gene.

17. A knockout rodent Znrf3 DNA construct comprising a selectable marker sequence or a reporter gene, or both, flanked by DNA sequences homologous to rodent Znrf3 genomic DNA, wherein when the construct is introduced into an embryonic rodent cell, the selectable marker sequence disrupts the rodent Znrf3 gene in the embryonic cell, and the rodent resulting from the embryonic cell exhibits increased bone mineral content (BMC) and bone volume compared to a wild type rodent in which the Znrf3 gene is not disrupted.

18. The knockout rodent Znrf3 DNA construct according to claim 17, wherein the construct comprises, 5′ to 3′:
a first rodent Znrf3 genomic DNA fragment;
a selectable marker sequence and/or a reporter gene; and
a second rodent Znrf3 genomic DNA fragment.

19. The knockout rodent Znrf3 DNA construct according to claim 18, wherein the selectable marker sequence is a neo cassette comprising a constitutive promoter.

20. A vector comprising the knockout rodent Znrf3 DNA construct according to any one of claims 17 to 19.

21. A conditional knockout rodent Znrf3 DNA construct comprising, in the 5′ to 3′ direction:
a first rodent Znrf3 genomic DNA fragment;
a first loxP site;
a first FRT sequence;
a reporter gene coding sequence;
a second FRT sequence;
a rodent Znrf3 cDNA coding sequence;
a second loxP site; and
a second rodent Znrf3 genomic DNA fragment.

22. The conditional knockout rodent Znrf3 DNA construct according to claim 21, wherein the rodent Znrf3 cDNA coding sequence is a rat Znrf3 cDNA coding sequence.

23. The conditional knockout rodent Znrf3 DNA construct according to claim 22, wherein the rat Znrf3 cDNA coding sequence comprises the ATG start codon through the stop codon of the wild type rat Znrf3 gene.

24. The conditional knockout rodent Znrf3 DNA construct according to claim 21, wherein the rodent Znrf3 cDNA coding sequence is a mouse Znrf3 cDNA coding sequence.

25. The conditional knockout rodent Znrf3 DNA construct according to claim 24, wherein the mouse Znrf3 cDNA coding sequence comprises the ATG start codon through the stop codon of the wild type mouse Znrf3 gene.

26. A vector comprising the conditional knockout rodent Znrf3 DNA construct according to any one of the claims 21 to 25.

27. A method of producing a genetically modified rodent, comprising:
transforming a rodent embryonic stem cell with a knockout construct comprising a selectable marker sequence and/or a reporter gene sequence, flanked by DNA sequences homologous to the endogenous rodent Znrf3 genomic DNA, thereby producing a transformed embryonic stem cell;
introducing the transformed embryonic stem cell into a rodent blastocyst; and
implanting the blastocyst comprising the transformed embryonic stem cell into a pseudopregnant female rodent, and allowing the blastocyst to undergo fetal development to term, to produce the genetically modified rodent;
wherein the genetically modified rodent is a heterozygous knockout rodent and exhibits increased bone mineral content (BMC) and bone volume compared to a wild type rodent.

28. The method according to claim 27, further comprising testing the produced genetically modified rodent to verify that its genome comprises a disrupted Znrf3 gene in at least one allele.

29. The method according to claim 27 or claim 28, wherein the genetically modified rodent is incapable of expressing an endogenous rodent ZNRF3 protein.

30. A method of producing a Znrf3 knockout rodent having a genome which is homozygous for a disruption of the rodent Znrf3 gene, the method comprising:
breeding a first heterozygous knockout rodent produced in accordance with any one of claims 27 to 29 with a second heterozygous knockout rodent to produce a progeny rodent; and
selecting a progeny rodent in which the disruption of the Znrf3 gene is homozygous.

31. The method according to claim 30, wherein the rodent is incapable of expressing an endogenous rodent ZNRF3 protein.

32. A progeny rodent produced by the method of any one of claims 27 to 31.

33. A method of determining the effect of an agent for treating high bone mineral density and/or bone mineral content, the method comprising:
administering the agent to a rodent that is heterozygous or homozygous for a Znrf3 gene knockout;
subjecting the rodent to a test to assess bone mineral density and/or bone mineral content; and
determining whether the agent has any effect on the bone mineral density and/or bone mineral content in the rodent.

34. A rodent model of increased bone mineral density and/or bone mineral content, wherein the rodent is heterozygous or homozygous for a Znrf3 gene knockout.

35. The rodent model according to claim 34, wherein the rodent is a rodent according to any one of claims 1 to 8.


SEQUENCE LISTING (5 sequences)

SEQ ID NO: 1 - DNA, 7199 bp, mus musculus
agagacaagtgactgtgtgtgtcagcgtccatctttcttcttgctctgcactggtcctggtcagaaatgttgaacttgactggcctccacctgcttccctttcctgggaccacagagccttgaccctgcatggggctatctggctatattttctccaagtgctttcaccatgcttcctcttgatcattggtgactatggctctcaaaattggtgcaaaatgaagattagcttttctctagtcatcacaatagctgtaccaacctctctttccaatagcttcacagtgagcattttgaccatagtatgatgcacccactaggcctatgtaataacaatgacgaagaggacttgtatgaatatggctgggtaggagtggtgaagcttgaacagccagaattggaccccaaaccatgtctcactgtcctgggcaaggccaagagagcagttcagcggggagctactgcagttatctttgatgtatctgaaaatccagaagccatcgaccagctaaaccaaggctcagaagaccctcttaagaggccagtggtgtatgtgaagggtgcagatgctatcaagctgatgaacattgtcaacaagcagaaagtagctcgagcaaggatccagcacctccctcctcgacaacccaccgagtactttgacatgggaattttcctggccttcttcgtcgtggtctccttggtctgcctcattctccttgtcaaaatcaagctgaagcaacgtcgtagtcagaattccatgaacaggctggctgtgcaggctctggagaagatggaaaccagaaagttcaactccaagagcaaggggcgccgggaaggaagctgtggggccctggacacactgagcagcggctccacatccgactgtgccatctgtctggagaagtacattgacggagaggagcttcgggtcatcccttgtactcatcggttccacaggaagtgtgtggatccatggctactgcagcaccacacctgcccccactgtcggcacaacatcatagaacaaaagggaaatccaggtgctgtttgtgtggagacaagcaaccttacacgtggtcggcagccaagagtgaccctaccggtacattacccaggccgtgtgcataggaccaatgccatcccagcctaccctactaggacaagtatggactcccatggcaaccctgtcacactgctaaccatggaccggcatggggagcagaacctctattctccacaaacacccacctatgtccgtggctacccacccctgcacctggaccacactctggcccctcatcgctgcagcctggaacaccgggcctactcaccagcccatcccttccgcaggcccaagttcagcagccgcagcttctccaaggcagcttgcttctcccagtatgagaccatgtaccaacactactacttccagggtctcagctacccagagcaggagggccagacaatacccagcgtcacacccaggggccaatcccgtgccttcccaccaagtggtgccagcagcctgctcttccccacaatggtacatgtggcccctccaacccacgtggagagtggcagcacatctagcttcagttgctatcatgggcaccgttctgtgtgtagcggctacctggctgactgtcctggcagcgatagcagcagcaacagctctggccagtgccgctgctcttccagtgactccgtggtggactgcacagaagtcagcaaccaaggtgtgtatgggagctgttccaccttccgaagctccctcagcagcgactatgaccccttcatctaccgcagccgggggcctgctgtgcaccttgagggctcccctccaccagaggagctcccagcaggacacagtcaaagtgctgggaggggcgagccatggctaggccctgcctctccctcaggggaccagctgtccacctgcagcctggagatgaactacagcagcaactcctcgctggaacccagagggcccaacagctctacctcagaagtggggctcgaggtttctcctggggctgccctggacctcaggaggacctggaaagggggtccagagggaccatcatgtgcttgctgcttcgagccccagcccttcccgccgggctctgggatagaaacaagtgcaggtggcagcagtttattcctgggtcctcggctcctcgaggactgcaaccctccaagtggggagccacagctggggagctcccagggcctgtatggtcttcactcagaccattatcctaggacagatggggtgaaatatgagggcttgccctgctgcttctatgaagagaagcaggtggcccacagtgctggcaggggcaatggctgctacactgaggactattcagtgagtgtacagtacacactcaccgaggaacccccacccagctgttatgcagggccccgggacctgagccagcgcatccccattattccagaggatgtagactgtgacttgggcttgccccaagactgccacgggatgcacaaccacagcccctggggtggggcactgagcttggacgttccccgactccactggagtctaggcacaacccgggaagaggaacaggctccatgttaccaagctgaggtgcagcctggctgctctccagaggaggcaggtgcttccagggctagcctctccagtgccccccaggacactcaggagtctcatgccctggctgctgaggcttcaggtgagaacagatgacagtagtatttgtataaggatccttgtctcagaccttttttgtttggcttcctctgtgcctttggcttctgtacttgatgactttcaaacattgcttttcagccctacccctcctcaaacgctatatccatgatgttggcatctcctcccatcagatgcttcatatcccaaaagcacatctcaattttattaactattctgcatctcctatggttccccttagtccatccctgtgtctatcaaagagcaaagtcctgacagaggtcactgcaattgtttgctctcccacttgggagaccttaatcacggtccttatactagaaatgctatcgtgacttttcagttacctttataagatgtctgtttttgtgtgttttctgtttggtttttgagacagggtttctttgtgtagaccaggctagtcttgaactcagatctctctgcttctgtctcctgagtactactgggattaaaggcatgcactaacactgcccagcataagatatcttctaactggagatgctccttttgatccagcctgtgtcttgttcctgcagcatgtcaggttcatttccatctccatcgagatccttcgatctcagctccacagccttcctttccccatcagtgtcactcccactgctatctcctaatcacctcaagccaggcacctcgtgggaagatctgtgatctgtgaggagtctcccctggccccctgggaactaattcacttaacttcagttctacctatgcttctgtgatatcttaggagattcagtataggcatcaagggagacctgctcaccctctgccccaacagctgttctcagtgtagacttgagctcctcaggaaaggagccattgctgccccctttatcacacagagccaaatcaccattgtgaactatcacatccaagggccctatggatgggctagggatgctcaaggaccttacctgatacgtactacccaaagcctgctgtgctggctctctccagacccttgatgactgttctccctctttctcccaggaccaggatctggcccaggcatcggtacgggagcttgagctcaaaaggaactcttatgtagaaatcgtgaactgtacggagacttcaaagtctggcttccttcaaaactactatacacacacacacacacacacacacacacacacacacacacacacacacacatatatatattttagctttgacaaacacaaaagtggtaataaagagagccctcctcaacccaaaatgtgagccacctgtggcatcccttcctctttaacaaaccaacagacaaaaatctcttgagtcctttgcctctttaataatgtgttgttctgttttgtaatgtgtgcttgggtttctgaggtgtgtgggattgagttctctgctttttttctaaaatattatatgtaaatgtaaaaagttatttaaatatatattttaaagaaccctaactaccaacttctgttgaaaaaaaaaatcactgctgcattaaatgaaccacatcatgtgtcaatactgttgtctccctggagggacctcaggcctttgaaaagctcagggttacacctgccttattgaaaatgaaccagaaacttgaagtaaaaagctccaaggaacaatacaaagctgcctcatcctttttcccttttttgaggcaaatctgaacttctgggcttctgggaagccacacctctcctgcccacacctgtaggcagcaatggaacaggagggaagtgggggcagtgtcaagctattactgcctaggccaactttattggagacagttctgcccattctcttctgttcctttggtaggtctttatcccagcagaggctgtggctgtgaccagccctgttactagggtctctcctgcactctgaccaccagtcaagggaagagacgctctcaccctgtggccttggagtcttttgtaaaatcattttgggcttcatgccatcaattgcaggggtaatttaaatggggtgtgcagggtggacacccaggaggaatgttgatttatggccttggtgtcctgtgggactagaggaacaagctctcctctcagtagcagtgcaccgctccttgaaaggcatgctgaggcacacttgtgcccatgggtctagggcagccacacccctcagctgttgcccccttaggttgccttcttgggggacatatgcttgaaccacaagagtgtactaagctccaattggaccagtcccccatccccagaaacaggcctctgcctggtggctgcagcggttcaccttgccagcagatgtctgctagcctgtctgcctgatgcactattatgtactgattctccacgagacagactatcagatagttcagacccaaagggtaggcttttgtatattttttccagcctctttttttttaaggggaagagttttttaaacccaaaccacttttggttgtttttgttattgtttttaagatgttttgtttatttttaaaagggagagagaattatttaaaatcacttcagggattgtaatcctttttattcaaaatagtccttgttcttaaaagtattttcttttccttagaggaaactaatcggcttggccctgtgtttgctaactcttctggtcacttttagtcattcatttagcagatgtctttttccatcaggaagagtttggccgagtgggtctgaaaactcggttacagaatggggaccacctgggttgccagagacagaaatgacccttcttcccagtcaggctcccaggaagtcatcccagggtccagattgcacatttgagatgatcagaatactttgttctttaaaacctcgtcctaggatgagggcatttgcttatttctatccagaatctaacaaaagaagtcccacgtcagatggcccagttatttgccctttaggaagtggccatagtgaaatttgttcttctgccggactctggaggtcggtcccttgaggctatgcccagtgcacctgttgctgagggtgtgagaagggcctaaactgttgacatgtaatcggggctgcctttctttctggggccaggagtagcagctgctaagaacagcagctttccttactgtgcaagggggggggtggaggcacagcttttaacatgaactgcaaagcacagcttctcattgttgagatgtttgttgtttctggttttccttttttttttttttttttttttttttttttgtgtgtgtgtgtctgtatgtgcatatttctccattggctgaaacaaaccccagaaagattttgcctttgtcctgcctggctagaaaaccatttctcagtactcatagatgagtagagaatttgaatgccactaggcagagttaaaaaaacaagttcaactctcctttgaaaaaatgctactagtgggtgacaggcacttgtaattgttgtagttttggactagtagaaacaaaagatttagagacaattatcatctttgtgttgccacatcagaccagaataaaacctatcttatgcaccatttaaatcagaagaaatagtgtctatgaagttctgagagccacagtgctctcttctgtgccctcttctggggaggggaagtggaaagggagtgtcagtagtgtgtggtgattgtatagccatggacaaggagcaccccactgtccactgggagcctgtgtgtggaatagcaagtgtgggcttgataccagccctgggtgtaagacagccatgccaacccaaaatctagaaccagatgaaaatggaagttaagtcattgtgtaccttgaggtgcctctgtggtcgtagctccagaagctgaccatccatcccctctcccccaggcccagaccgctgtatccgtgtccctctttagtcccgtggttcaggcctttccctatgtgcttctgctctcaaagtccacacaggactgatgcttttaaaatgtggtctgtaaaagaatttgaaagtatcatcttttaaactgacctgcatttttctattccttacataaactttttgtatcgtgttggtctaatgacactattttaattttttttttatttctcccatagcacttaatattttgtaaagatttttttgtgataaaatggtctaagggctctttttccacattaccattttaaaaacttttaaaagctagaaaacaacttatgtatattctgtatatgtatagcagcacatttcatttatggaaatatgttctcagaatatttatttactaatatatttatcttaagccatgtcttatgttgagagtgtgacattgttgaaataatcattgaaaatgactaacaaggccctgtaaatacatgataattgcacacagattttacatatttgcagaccaaaaatgatttaaagcaagttgtagtcgtctatgattttgtaacaaattgtacaaatgactgtaaaaaaaaatacaattttatcaagta

SEQ ID NO: 2 - DNA, 2465 bp, rattus norvegicus
gtcatttctccacccctataaaatatctatttttaggggttttattgtgttctcttcagtgctcacctctctgtaatgaattgaaataaccattggattgaaaaactattctgtgccataaagtctgaagttttgaagtggcttttgcttcatatgtaataagaaatgcatgaagtgggttagatcagtctgtggctagcctgttgatattttcttgccattttgtcttttagatgcacccactaggcctatgtaataacaatgacgaagaggacttgtacgaatatggctgggtaggagtggtgaagctcgaacagccagaattggaccccaaaccatgtctcactgtcctgggcaaggccaagagagcagttcagcggggagctactgccgttatctttgacgtatctgaaaacccagaagccatcgaccagctaaaccaaggctctgaagacccccttaagaggccagtggtgtatgtgaagggtgcagatgctgtcaagctgatgaacattgtcaacaagcagaaagtagctcgagcaaggatccagcacctccctcctcgacaacccaccgagtactttgacatggggattttcctggcattcttcgtcgtggtctccttggtctgcctcattctccttgtcaaaatcaagctgaagcagcgtcgtagtcagaattccatgaacaggctggccgtgcaggctctggagaagatggaaaccagaaagttcaactccaagagcaaggggcgccgggaaggaagctgtggggccctggatacacttagcagcggctccacatccgactgtgccatctgtctggagaagtacattgatggagaggagcttcgggtcatcccgtgtacccatcgattccacagaaagtgtggggatccatggctactgcagcatcatacccgtccccattgtcggcacaacatcataggtaactgctacaggcctcgtcacaccctgacctacaaagatgacaagtgcgtgctgcacgctaggtggagaatcagaagatattggtttacgcgtgcgcgcccacctgcccacctgcgaccaactcatgcccacactctggcccctcatcgctgcagcctggaacaccgggcctactcaccagcccatcccttccgcaggcccaagttcagcagccgcagcttctccaaggcagcttgcttctcccagtatgagaccatgtaccaacactactacttccagggtctcagctacccagagcaggagagccagccggtacctagcgtcacacccaggggccagtcccgtgccttcccaccaagtggtggcagcagcctgctcttccccaccatggtgcatatggcccctccagcccatgtggagagtggcagcacgtctagcttcagttgctaccacgggcaccgttctgtgtgtagtggctacctggctgactgtcctggcagcgatagcagcagcaacagctctggccagtgccgctgctcttccagtgactctgtggtggactgcacagaagtcagcaaccaaggtgtgtatgggagctgctccaccttccgaagctccctcagcagcgactatgaccccttcatctaccgcagccgggggcctgctgtgcaccttgagggctccccgccaccagaggagcttccagcagcccacaatcaaggtgctgggcggggtgagccatggctaggccctgcctctccctcaggggaccagctgtccacctgcagcctggagatgaactacagcagcaactcctcgctggaacccagagggcccaacagctctacctcagaagtggggctcgaggtttctcctggggccgccctggacctcaggaggacctggaaagggggcccagagggaccatcatgtgcttgctgctttgagccccagcccgtcacgctgggctctgggataggaacaactgcaggtggtagcagcttattcctgggtccccgactccttgaggactgcaaccctgcaagtggggagccacaactagggagctcccagggcctgtatggccttcactcagaccattatcctaggacagatggggtgaaatatgagggcctgccctgctgcttctatgaagagaagcaggtggcccacagtgctggcaggggcaatggctgctacactgaggactattcagtgagtgtgcagtacacactcaccgaggaacccccacccagctgttatgcaggggcccgggacttgagccagcgcatccccattattccagaggatgtagactgtgacttgggcttgccccaagactgccaagggaggcacagccacagcccctggggtggagcactgggcttggatactaaaaaatgctttgtggacaacggaaaaagacggaacaggctccacctgttaccagctgacggtgcagctgctgttccctacttctgcctccggagtactgggattaaaggcgtgcactaa

SEQ ID NO: 3 - protein, 809 aa, mus musculus
MMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHLPPRQPTEYFDMGIFLAFFVVVSLVCLILLVKIKLKQRRSQNSMNRLAVQALEKMETRKFNSKSKGRREGSCGALDTLSSGSTSDCAICLEKYIDGEELRVIPCTHRFHRKCVDPWLLQHHTCPHCRHNIIEQKGNPGAVCVETSNLTRGRQPRVTLPVHYPGRVHRTNAIPAYPTRTSMDSHGNPVTLLTMDRHGEQNLYSPQTPTYVRGYPPLHLDHTLAPHRCSLEHRAYSPAHPFRRPKFSSRSFSKAACFSQYETMYQHYYFQGLSYPEQEGQTIPSVTPRGQSRAFPPSGASSLLFPTMVHVAPPTHVESGSTSSFSCYHGHRSVCSGYLADCPGSDSSSNSSGQCRCSSSDSVVDCTEVSNQGVYGSCSTFRSSLSSDYDPFIYRSRGPAVHLEGSPPPEELPAGHSQSAGRGEPWLGPASPSGDQLSTCSLEMNYSSNSSLEPRGPNSSTSEVGLEVSPGAALDLRRTWKGGPEGPSCACCFEPQPFPPGSGIETSAGGSSLFLGPRLLEDCNPPSGEPQLGSSQGLYGLHSDHYPRTDGVKYEGLPCCFYEEKQVAHSAGRGNGCYTEDYSVSVQYTLTEEPPPSCYAGPRDLSQRIPIIPEDVDCDLGLPQDCHGMHNHSPWGGALSLDVPRLHWSLGTTREEEQAPCYQAEVQPGCSPEEAGASRASLSSAPQDTQESHALAAEASGENR

SEQ ID NO: 4 - protein, 743 aa, rattus norvegicus
MHPLGLCNNNDEEDLYEYGWVGVVKLEQPELDPKPCLTVLGKAKRAVQRGATAVIFDVSENPEAIDQLNQGSEDPLKRPVVYVKGADAVKLMNIVNKQKVARARIQHLPPRQPTEYFDMGIFLAFFVVVSLVCLILLVKIKLKQRRSQNSMNRLAVQALEKMETRKFNSKSKGRREGSCGALDTLSSGSTSDCAICLEKYIDGEELRVIPCTHRFHRKCGDPWLLQHHTRPHCRHNIIGNCYRPRHTLTYKDDKCVLHARWRIRRYWFTRARPPAHLRPTHAHTLAPHRCSLEHRAYSPAHPFRRPKFSSRSFSKAACFSQYETMYQHYYFQGLSYPEQESQPVPSVTPRGQSRAFPPSGGSSLLFPTMVHMAPPAHVESGSTSSFSCYHGHRSVCSGYLADCPGSDSSSNSSGQCRCSSSDSVVDCTEVSNQGVYGSCSTFRSSLSSDYDPFIYRSRGPAVHLEGSPPPEELPAAHNQGAGRGEPWLGPASPSGDQLSTCSLEMNYSSNSSLEPRGPNSSTSEVGLEVSPGAALDLRRTWKGGPEGPSCACCFEPQPVTLGSGIGTTAGGSSLFLGPRLLEDCNPASGEPQLGSSQGLYGLHSDHYPRTDGVKYEGLPCCFYEEKQVAHSAGRGNGCYTEDYSVSVQYTLTEEPPPSCYAGARDLSQRIPIIPEDVDCDLGLPQDCQGRHSHSPWGGALGLDTKKCFVDNGKRRNRLHLLPADGAAAVPYFCLRSTGIKGVH

SEQ ID NO: 5 - DNA, 4134 bp, mus musculus
accgagtactttgacatgggaattttcctggccttcttcgtcgtggtctccttggtctgcctcattctccttgtcaaaatcaagctgaagcaacgtcgtagtcaggtaaccctaggagtgacctgtgaaccccattaccctgttcctgatgacagctggggaaccaggtgtagtggcacactcttttaatcccagtacttgggaggcagaggcaggtgggtctccgagttcaaggccagcttagtctacatagtgagttccagtagagtcagggctgctacgcagagaaaccctgtctctcaaaacttaaaaacaaaaaaaagaaaagaaagttagggatctgaccagcctgttgtctatggcacccaaactacctcagtggcttggagggtaccagacctcaagtactgagcccccatgtggagaagagcaggcttgtccacaaaggcctcagcattgagggtgttccaaactagtattttgagggaactgtttaaataaatgctgctttggagcatgagaccttgctcctttagtagatctgaggttggtagaaataaagacggtcaggctcctaggtcttatgatgaaatttgcctctgccaggtccatctgttaaatgttgatgaacaggtagctatagctagtactctagcaatcccatcactgtggcttacaagagagaagtacctataggcagaggatggtgagagggaaagcaggagactagactttgagatatgtggcggtgtgacatcttgggagaaagggcaccttagatagtgactgtcaaaaggaggttacagttgtcaggagggttgaggggccaccaccagcaagacaggactaggggttagtatttgtgcactcacaccgcatgtcgggtgctgagcactcacgtctcatttccctggcttccttcaacaaggagggacatttgacctagagccttctaaaacccaatgttgcacagaattaagtatcagagacagggaattgcctttcctcttgaaggcaagactttgtagtagagtcagtgtcgtgggcactgttggtctaggaaatacagtgcaatatgaatttggaacagctcctcagggatctgtttgacctaagtgagattagttttgtctgtcacagtgtcatgagtgccatggcctggggtatgcctaccactgataatgtgtcccaacactgaccatgctctgccatgcagaattccatgaacaggctggctgtgcaggctctggagaagatggaaaccagaaagttcaactccaagagcaaggggcgccgggaaggaagctgtggggccctggacacactgagcagcggctccacatccgactgtgccatctgtctggagaagtacattgacggagaggtagctgcaggatcagggcctgccttgggtgggaagggatgtacagaggccccaggaggtgctggggactaccatgaccatgactagagttctccacaacctagattcagtgcgtaagcttctaaaggcatttcttcctggggctgggaaaacacaatactctatagatggtctagctaataagtacatggttggccagggtagtagcacatgccagtagttttagtgcttgagagactactcaaaagaagtgtgagtttaagatcccggctttgccaaaatgcacatggttgaacagaaaatatatagatgtaagatcaagttgcttactttgctttgaagaactggttaagtaatgagtttacttggagaaagtctagaaatgtgatgccttttgttttttcctgtagctctttattaagagttgcaagtatcaggaacagcacagaaccaccattcacatattttgctacctttatgtctgtcttcagatggacaggtacatacatagtggagaggaaactgcctgagagtccaggccttgaacagcccctggataagatcgacctcccccctgatgtgtaggttgctgggtggaagtatgtttgtacacaggcccttggatgatacgcttgtgtagactgtacgtacagactcctgggctctaacctagcccaggcagattcttggtttgtgaacttgacgagtgacttcattctgtgtctgcgcttgtgacaccaaccagcatactagattctcacaaacactatttaggtgagaagatacaagcagtctttttatttttcttttaagatttatgtatgtatatgagcactctaatttgcgtgtacacctgcgtgccaggatcccattacagatagttgtgagtcaccatgtggttgctggaaattgaactcgggacctctgaaagagcagccactaagccatctctccaaccctgatacaggcattctaaagcaccatattggctatttattttaatatcactgtatcagactggtgggaaaaggggaaaaaaaagaaaagaaaaggaataagaacaagtcagtaggatacttcggttttctttcaaattggctgctttccttagaaacaatgtggccctatcaagtgttccttttctacttgacctcattcaatggtacttagaggcaaccatccagtagttctcaaccttcctaatgctgtgaccctttaatacagtttctcatattgtggtgacccccaaccgtaagataattttcatgactactaaataactgtaattttgttattgttatgaaacgtaatatacaaagctgtattttccgataattttttgaggtcttgacccataggttgggaaccacagatctatacctatatagatagcctcgggtatttgttactctccctgcaaaccctttgttccctggcctgtggctttggacttagatgtgcccccagttcctggaaaggaatctgcttccttatgctccttgtcttttagccctgatagctgtggttaggacagatgaattggccagtttccttctccttgtgggccatacttcaagggatgagactggcctttagagttagggaagagagtgggaagtaccctgactttccatttctgtaacaaacacttgcagtaataaacaggtaaagagcaaaggtttgtttaaacacatagttttagacttttgttttatttttaatgattttatttggtttatgtatatgagtgctctatctacatgtacacctgcgtgccagaagagggcatcagatcccattacagatggttgtgagtcaccatgtggtagctgggagttgaactctggacctctggaagagcagccaatgctcttatcttatgaacggtctctccagcctagttttggacttttaacccacacacttaccacaatcctttattaggcttatagaaaggcagcatatcatggcaagaatatgtgatggctaggactggaaaggtgcctcagtggttatgagcacatgcctatcttggagaggatctgacttcagttcccagtaccctcttctattctcctcaggtaactacacacacatgcacagatatcaagcatgcatacacataattcaaagtaaaaattaaggcagccatgcagccttgtaagtcttgtaatataccgccatgaatttctcagaacagtggaccttcttcttttcttctatctcttttctaagcatgctttctctcatgctgtagacttccttgctctttgtccaattatctgactctaggccagcttaacttaacttaacttaagtggcatgactctcttccctcatccattgtccttggagcaactgatgaggcatactgcttgcctggcctggaagttttctctttatcactaataaaattatccaggaggacacaagaagaggaagaaaggaaaccaggcccacttggcacccttcccttgtactgccgagctaggaacacttaggatccatcttacagcagggggacaccagcacagtgcacaggctgcctgttccccgttggctccattgaaagaagcacgtgggtgaggctgaaggcagaagtaccctctctccagttcacccttcggtctgtgttctttatctctctctgcaggagcttcgggtcatcccttgtactcatcggttccacaggaagtgtgtggatccatggctactgcagcaccacacctgcccccactgtcggcacaaca